(19) United States
(12) Patent Application Publication
(10) Pub. No.: US 2024/0383953 A1
(43) Pub. Date: Nov. 21, 2024

(54) NOVEL NANOMATERIALS FROM NANOG PRION-LIKE REPEATS

(71) Applicant: BAYLOR COLLEGE OF MEDICINE, Houston, TX (US)

(72) Inventors: Josephine C. Ferreon, Houston, TX (US); Allan Chris M. Ferreon, Houston, TX (US)

(73) Assignee: BAYLOR COLLEGE OF MEDICINE, Houston, TX (US)

(21) Appl. No.: 18/690,725

(22) PCT Filed: Sep. 12, 2022
(86) PCT No.: PCT/US2022/043216

§ 371 Date: Mar. 9, 2024

Related U.S. Application Data
(60) Provisional application No. 63/243,310, filed on Sep. 13, 2021.

(51) Int. Cl. C07K 14/47; A61K 38/00; C07K 7/06; C07K 7/08

(52) U.S. Cl. CPC C07K 14/4702; C07K 7/06; C07K 7/08; A61K 38/00

(57) ABSTRACT

Embodiments of the present disclosure pertain to isolated peptides of any one of SEQ ID NOS: 1-14, derivatives thereof, analogs thereof, homologs thereof, and combinations thereof. The isolated peptides may be in aggregated form, fibrillated form, in the form of three-dimensional hydrogels, or combinations thereof. Additional embodiments of the present disclosure pertain to methods of delivering the isolated peptides of the present disclosure into cells by exposing the cells to the isolated peptides and/or nucleotide sequences that express them. In some embodiments, the exposing results in the conversion of the cells to pluripotent stem cells. In some embodiments, the exposing occurs in vitro. In some embodiments, the exposing occurs in vivo in a subject by administering the isolated peptides and/or nucleotide sequences of the present disclosure to the subject.

### CROSS-REFERENCE TO RELATED APPLICATIONS

This application claims priority to U.S. Provisional Patent Application No. 62/243,310, filed on Sep. 13, 2021. The entirety of the aforementioned application is incorporated herein by reference.

### STATEMENT REGARDING FEDERALLY SPONSORED RESEARCH

This invention was made with government support under R01 GM122763 awarded by the National Institutes of Health. The government has certain rights in the invention.

### BACKGROUND

The master pluripotency factor NANOG potently self-assembles through its prion-like WR domain, is a master epigenetic re-programmer, and key to stem cell pluripotency. Furthermore, NANOG controls entry to stem cell pluripotency. In particular, human NANOG expression is necessary to reset human stem cells to pluripotent ground state with unlimited self-renewal potential similar to mouse embryonic stem cells (ESC). Various embodiments of the present disclosure aim to maximize the aforementioned benefits of NANOG.

### SUMMARY

In some embodiments, the methods of the present disclosure pertain to at least one isolated peptide. In some embodiments, the at least one isolated peptide includes, without limitation, any one of SEQ ID NOS: 1-14, derivatives thereof, analogs thereof, homologs thereof, and combinations thereof. In some embodiments, the isolated peptides of the present disclosure may be in aggregated form, fibrillated form, in the form of three-dimensional hydrogels, or combinations thereof.

Additional embodiments of the present disclosure pertain to methods of delivering the isolated peptides of the present disclosure into cells. In some embodiments, the methods of the present disclosure include exposing the cells to the isolated peptides of the present disclosure and/or nucleotide sequences that express them. In some embodiments, the exposing results in the conversion of the cells to pluripotent stem cells. In some embodiments, the exposing occurs in vitro. In some embodiments, the exposing occurs in vivo in a subject by administering the isolated peptides and/or nucleotide sequences of the present disclosure to the subject.

### DRAWINGS

FIGS. 1A-1B illustrate that the WR domain of NANOG limits its solubility. FIG. 1A illustrates various NANOG constructs, including NTD (N-terminal domain), DBD (DNA-binding domain), CTD (C-terminal domain), and WR (tryptophan repeat domain). FIG. 1B provides data regarding the solubility of NANOG constructs. Solubility was determined from the ratio of proteins in the supernatant relative to the pellet fractions after centrifugation (n=3; 6 μM for all constructs except for 100 μM NANOG NTD).

FIGS. 2A-2C provide data related to the characterization of NANOG NTD and CTD domains. FIG. 2A provides a CD spectrum of NANOG NTD (100 μM), which shows a random coil signature. FIG. 2B provides a 2D NMR ¹⁵N-HSQC spectra of ¹⁵N NANOG NTD (500 μM), showing limited peak dispersion in ¹H dimension. FIG. 2C shows a 1D ¹H NMR spectra of NANOG CTD at 10 μM (256 scans; ˜10 min), showing the absence of strong NMR backbone amide and tryptophan side chain peaks.

FIG. 3 provides a NANOG predicted disorder. Domain organization of NANOG and disordered region prediction were generated by PONDR VS-L21.

FIGS. 4A-4I show that NANOG CTD displays prion-like behavior. FIG. 4A shows smFRET histograms showing number of events vs FRET efficiencies (E_{FRET}) of NANOG CTD (conjugated with AF488-AF594 FRET pair) with increasing GdnHCl concentration (top to bottom). FIG. 4B shows E_{FRET} as a function of Gdn HCl concentration. FIG. 4C shows CD spectra of 2 μM NANOG CTD. FIG. 4D shows CTD aggregation kinetics monitored by ThT fluorescence. Graphs fitted to an exponential function y=Ac^{Rx} with time constant (1/R) of 4.8±0.2 min and 9.5±0.1 min for 1.2 μM (n=9) and 2.4 μM (n=6) CTD, respectively. FIG. 4E shows a CTD can form gel-like condensates at low 5 μM concentration. FIG. 4F shows an SDD-AGE gels (Stain-free and ThT-stained), showing WR and CTD as high MW ThT-positive complexes with BSA as negative controls. FIG. 4G shows scanning electron micrographs of NANOG CTD gel, showing porous, fibril-like networks. FIG. 4H shows CD spectra of CTD alternating tryptophan mutants (W1357A and W468A) with disrupted/reduced β-sheet propensity. FIG. 4I shows homology modeling of selective NANOG WR repeats with known prion proteins.

FIGS. 5A-5I show that NANOG oligomerizes at low nM concentrations. FIG. 5A shows chemical crosslinking of endogenous NANOG in H9 ES cells. FIG. 5B shows chemical crosslinking of NANOG variants (WT, ΔWR, and W8A) expressed in HEK 293T cells. FIG. 5C shows UV-detected SEC of MBP-fused NANOG WT (˜300 nM), W8A (˜1.5 μM), and h6g-NANOG:Skp complex with MW calibration standards. FIG. 5D shows fluorescence-detected SEC of GFP-tagged NANOG WT/W8A (˜10 nM) and h6g-eGFP. NANOG WT elutes in the void volume corresponding to high MW aggregates. FIG. 5E shows DSSO chemical crosslinking of purified GFP-tagged NANOG WT/W8A (2.5 nM). FIGS. 5F1-3 shows autocorrelation FCS curves (top) and corresponding derived diffusion coefficients (bottom) of (i) h6g-cGFP and GFP-tagged NANOG WT/W8A (˜10 nM) constructs in HEK 293T mammalian cell lysates, (ii) purified GFP-tagged NANOG WT/W8A constructs (˜5 nM), and (iii) refolded AF488-conjugated NANOG WT/W8A (˜10 nM) and SOX2 proteins (˜10 nM). The times shown (5 min and 4 hr) represent incubation periods prior to FCS data collection. HEK 293T cell lysates were treated with benzonase to prevent DNA-protein interactions that could dramatically affect NANOG's diffusion properties. Error bars represent SD; n=3-6 across two independent replicates; *p<0.05 and **p<0.01 significance levels. FIG. 5G shows photocount histograms (PCH) of NANOG WT (black), W8A (red), and h6g-cGFP control (green). FIG. 5H shows photon bursts distribution of NANOG WT vs W8A mutant. FIG. 5I shows binned burst histograms (multiples of the average photon rates or counts per second) of WT vs W8A mutant.

FIGS. 6A-6G provide data related to intermolecular DNA bridging through NANOG assemblies. FIG. 6A shows representative fEMSA of NANOG WT vs W8A mutant with 5 nM Gata6-AF647. FIG. 6B shows a NANOG WT/W8A oligomer population (i.e., band intensities in fEMSA wells). FIG. 6C shows fractions of unbound DNA (fEMSA white rectangles shown in FIG. 6A) with 125 and 250 nM NANOG WT/W8A (n=3). FIGS. 6D1-3 shows smFRET of ˜100 pM each Gata6-AF488 and Gata6-AF647 intermolecular diffusion in (i) the absence or (ii-iii) presence of 250 nM NANOG W8A and WT mutant. The peak at E_{FRET} ˜0 corresponds to AF488-conjugated unbound/bound DNA. FIGS. 6E1-4 show representative cross-correlation FCCS curves of Gata6-AF488 and Gata6-AF647 with various concentrations of WT ((i) 0 nM; (ii) 63 nM; (iii) 875 nM) and W8A mutant ((iv) 875 nM). FIG. 6F shows number of cross-correlated particles (N_{ad}) per μm³ volume (left y-axis) and diffusion coefficients of WT/W8A-DNA complexes (right y-axis) in relation to protein concentrations. N_{ad} and diffusion coefficients were derived and calculated from FCCS fits (Methods). FIG. 6G shows a model for how NANOG can help shape the pluripotent genome. Through prion-like assembly, NANOG can initiate intragenomic (promoter-enhancer) contacts, as well as connecting distant intergenomic loci to form superenhancer clusters with other TFs and coactivators (green/yellow).

FIGS. 7A-7H provide data and illustrations related to the fibrillation propensities of NANOG WR-derived peptides. FIG. 7A illustrates NANOG domain organization and the human and mouse NANOG WR-derived peptides used in Example 2. FIG. 7B shows amino acid type distributions for the peptide constructs. FIG. 7C shows peptide solubilities determined from the ratios of supernatant to pellet fractions after centrifugation (mean and SD of three independent replicates; 0.1 mg/mL and 0.2 mg/mL. top and bottom panels respectively). FIG. 7D shows WR peptide (0.2 mg/mL) aggregation kinetics monitored by ThT fluorescence after 20 min equilibration. Right panels show aggregation kinetics for peptides 1-3 w (top) and 1-4 w (bottom) after 1 min equilibration. Similar results were obtained from two independent experiments. FIG. 7E shows ¹H 1D NMR spectra of the tryptophan side chain region of peptide 1-4w as a function of time. Similar results were obtained from two independent experiments. FIG. 7F shows changes in NMR peak intensity as a function of time. Data points were fitted to an exponential decay function with a half-life of 360±20 s (mean±SD). FIG. 7G shows scanning electron micrograph (SEM) of peptide 1-4w. Similar results were obtained from two independent experiments. FIG. 7H shows WR 1-4w readily forms a hydrogel at ˜2 mg/mL (0.2% w/v) in TBS buffer (25 mM Tris, 140 mM NaCl, pH 7.4). Similar results were obtained from two independent preparations.

FIGS. 8A-8F show that NANOG peptide mimetic 1-4wRK bridges DNA. FIG. 8A shows the WR 1-4wRK peptide sequence. Basic residues, 3 Arg and 2 Lys, were incorporated for nucleic acid recognition. FIG. 8B shows fEMSA of 1-4wRK peptides with dsDNA GATA6-AF488 and GATA6-AF647 (left), ssDNA (TG) 6-AF488 (middle) and ssRNA (UUAGGG)₄-AF488/AF594 (right). Similar results were obtained from two independent experiments. FIG. 8C (left panels) show photon bursts distribution of dsDNAs alone (GATA6-AF488 and GATA6-AF647; top) or with 7.5 μM (0.03 mg/mL) 1-4wRK peptide (bottom). Middle panels of FIG. 8C show FCCS curves for corresponding photon fluctuations shown in the left panels. Right panels of FIG. 8C shows boxed and color-coded Regions I-III from the bottom leftmost panel FIG. 8C, which were analyzed separately to generate correlation curves. Similar results were obtained from three independent experiments. FIG. 8D shows binned burst histograms (multiples of the average photon rates or counts per second) as a function of peptide concentrations 0-30 μM or 0-0.15 mg/mL. Similar results were obtained from three independent experiments. FIG. 8E shows a fraction of species that display photon bursts (determined as 10× deviation from average fluctuation) as a function of peptide concentrations. Error bars were calculated from three independent measurements. FIG. 8F shows co-partitioning of 1-4wRK (70 μM or 0.27 mg/mL) with fluorescent proteins (h6GeGFP and h6GmCherry; 5 μM or 0.18 mg/mL). Similar results were obtained from two independent experiments.

FIGS. 9A-J shows modulation of WR peptide material states. FIG. 9A shows WR-derived and TAT-derived peptide sequences. FIG. 9B shows pie charts illustrating the composition of hardening (aromatic and polar) versus softening residues (charged and structure breakers) of WR-derived peptides. FIG. 9C shows relative peptide solubility (mean±SD of 3 independent measurements) at 0.1 mg/mL (top) and 0.2 mg/mL (bottom). FIG. 9D shows ThT fluorescence emission spectra of different WR peptides. ThT binding to amyloid β-sheet emits maximally at ˜490 nm. Similar results were obtained from 2 independent experiments. Top portion of FIG. 9E shows fluorescence microscopy images showing colocalization (merged; yellow) of 1-4wRRK (green) with dsDNA GATA6-AF647 (red). Middle portion of FIG. 9E shows fluorescence recovery after photobleaching (FRAP) images of targeted areas (white arrows) monitoring WR 1-4wRRK-AF488 (green) or GATA6-AF647 (red) clusters. Bottom portion of FIG. 9E shows average FRAP curves of WR 1-4wRK-AF488: GATA6-AF647 clusters (n=3 and n=6 puncta across 2 independent replicates, respectively). Top portion of FIG. 9F shows fluorescence microscopy showing colocalization (merged; yellow) of 1-3wRRK (green) with dsDNA GATA6-AF647 (red) in LLPS droplets. Middle portion of FIG. 9F shows FRAP images of WR 1-3wRRK-AF488 (green): GATA6-AF647 (red) droplets (bleached at positions with white arrows). Bottom portion of FIG. 9F shows average FRAP curves (n=11 droplets across 2 independent replicates). FIG. 9G shows fEMSA of 1-3wRRK vs. TAT peptides with dsDNA GATA6-AF647/AF488 (5 nM each). Similar results were obtained from 2 independent experiments. FIG. 9H shows fluorescence polarization binding measurements of TAT and WR peptides with GATA6 dsDNA. Data were fitted to a Hill binding equation with n as the degree of cooperativity and binding constant Kₐ, (mean±SD, n=3 independent measurements. FIG. 9I shows FCCS curves of TAT and WR peptides (1-3wRRK and 1-4wRK) at 0.9 μM. Similar results were obtained from 3 independent experiments. FIG. 9J shows LLPS diagrams of TAT and WR peptides (1-3wRRK and 1-4wRK). LLPS positives (dark circles) are classified by average turbidity or UV Abs_{350 nm}>0.07 (3 independent measurements). 1-4wRK aggregates even without DNA at 0.2 mg/mL, exhibiting positive turbidity readings at low DNA concentration.

FIGS. 10A-H show translational applications of NANOG WR peptides. FIG. 10A shows bright field (left panels) and fluorescence microscopy images (right panels) of HEK 293T cells transfected with ssDNA-AF488 (green; 2.4 ng/μL or 0.5 μM) alone or with WR 1-3wRRK (66 ng/μL or 20 μM). Similar results were obtained from 2 biological replicates. FIG. 10B shows fluorescence microscopy images of HEK 293T cells (nuclei, blue) transfected with 3486 bp DNA plasmid (pmaxGFP; 10 ng/μL, 4.3 nM) and TAT (118.7 ng/μL or 76.4 μM; left panel) or WR 1-3wRRK peptide (125 ng/μL or 38 μM; right panel). Similar results were obtained from 2 biological replicates. FIG. 10C shows differential interference contrast (DIC) microscopy images of HEK 293T cells transfected with β-galactosidase (10 ng/μL, 84 nM) alone or together with 1-3wRRK peptide (17.4 ng/μL or 5.2 μM). Blue color represents presence of β-galactosidase in the cells. Similar results were obtained from 2 biological replicates. FIG. 10D shows fluorescence microscopy images of HEK 293T cells (nuclei, blue) transduced with lentiviral particles (1×10⁶ TU, carrying mTurquoise2 gene; green) alone or together with 1-3wRRK peptide (125 ng/μL or 38 μM). Similar results were obtained from 2 biological replicates. FIG. 10E shows fluorescence microscopy images of BJ fibroblast cells (nuclei, blue) plated on WR 1-4w-coated dishes (1 mg/mL or 300 μM). Right panel of FIG. 10E is a bright field microscopy image after MTT staining, showing live cells in the condensed areas. Similar results were obtained from 2 biological replicates. FIG. 10F shows enzymes (Alkaline phosphatase (AP) and horse radish peroxidase (HRP)) were mixed with WR-based hydrogels (combination of 1-4w and 1-3wRRK peptides). Partitioning of AP enzyme in the hydrogel was verified by SDS-PAGE (left panel), showing negligible AP in the hydrogel supernatant and excess unpolymerized WR peptides. Input AP was shown for comparison (left lane). Right panel of FIG. 10F shows positive catalytic activities of the enzymes, which were determined by colorimetric assays. Pinkish red color results from the generation of the fluorescent resorufin compound in the presence of HRP enzymatic activity. Yellow color results from the removal of the substrate BBTP's phosphate group in the presence of alkaline phosphatase. Similar results were obtained from 2 independent experiments. FIG. 10G shows kinetic release of the drug Doxorubicin (500 μM, orange) from WR-based hydrogels (combination of 1-4w and 1-3wRRK peptides). Error values represent mean±SD for 3 independent experiments. FIG. 10H shows a schematic of the nano-to-macroscale translational applications for NANOG-inspired biomaterials.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The master pluripotency factor NANOG potently self-assembles through its prion-like WR domain, is a master epigenetic re-programmer, and key to stem cell pluripotency. Furthermore, NANOG controls entry to stem cell pluripotency. In particular, human NANOG expression is necessary to reset human stem cells to pluripotent ground state with unlimited self-renewal potential similar to mouse embryonic stem cells (ESC).

Full-length human NANOG behaves like a functional amyloid. At low nM concentrations, NANOG readily self-assembles through its WR domain into large oligomers. The assembly process enables NANOG to function as a master transcription factor and efficiently recruit DNA.

Furthermore, NANOG controls entry to stem cell pluripotency. In particular, human NANOG expression is necessary to reset human stem cells to pluripotent ground state with unlimited self-renewal potential similar to mouse embryonic stem cells (ESC). Various embodiments of the present disclosure aim to maximize the aforementioned benefits of NANOG.

In some embodiments, the present disclosure pertains to at least one isolated peptide. In some embodiments, the at least one isolated peptide includes a sequence resembling the prion-like domain of NANOG. Further embodiments of the present disclosure pertain to methods of delivering the isolated peptides of the present disclosure into various cells for various purposes.

For instance, in some embodiments, the methods of the present disclosure pertain to a method of delivering the isolated peptides of the present disclosure to cells by exposing the cells to the isolated peptides and/or nucleotide sequences that express the isolated peptides.

The isolated peptides and delivery methods of the present disclosure can have numerous embodiments. For example, in some embodiments, the isolated peptides of the present disclosure can have various prion-like domains, be in various forms, and include various sequences resembling the prion-like domains of NANOG. Furthermore, the delivery methods of the present disclosure can deliver the isolated peptides of the present disclosure into various cells for various purposes.

### Isolated Peptides

As set forth in further detail herein, the isolated peptides of the present disclosure can have numerous embodiments. For example, in some embodiments, the isolated peptides can have various prion-like domains, be in various forms, and include various sequences resembling the prion-like domain of NANOG. Furthermore, the isolated peptides of the present disclosure can have various origins and applications.

### Prion-Like Domains

The isolated peptides of the present disclosure can include various prion-like domains. For example, in some embodiments, the prion-like domain is the WR domain within the prion-like domain of NANOG. In some embodiments, the prion-like domain is a segment of the WR domain of NANOG. In some embodiments, the prion-like domain is the entire WR domain of NANOG.

### Peptide Forms

The isolated peptides of the present disclosure can have numerous forms. For instance, in some embodiments, the isolated peptide is in aggregated form. In some embodiments, the isolated peptide is in fibrillated form. In some embodiments, the isolated peptide is in the form of a three-dimensional hydrogel. In some embodiments, the isolated peptide is in the form of a prion-like nanomaterial.

In some embodiments, the isolated peptide is in the form of peptides dissolved in solvents or solutions. In some embodiments, the peptides may be in homogeneous or heterogeneous forms.

The isolated peptides of the present disclosure can include various sequences. For instance, in some embodiments, the sequence shares at least 95% identity with a sequence within the prion-like domain of NANOG. In some embodiments, the sequence shares at least 90% identity with a sequence within the prion-like domain of NANOG. In some embodiments, the sequence shares at least 85% identity with a sequence within the prion-like domain of NANOG. In some embodiments, the sequence shares at least 80% identity with a sequence within the prion-like domain of NANOG.

In some embodiments, the isolated peptides of the present disclosure include, without limitation, WSNQTK (SEQ ID NO: 1); WSNQTWNNSTK (SEQ ID NO: 2); WSNQTWNNSTWSNQTK (SEQ ID NO: 3); WSNQTWNNSTWSNQTQNIQSWSNHSK (SEQ ID NO: 4); ASNQTANNSTASNQTQNIQSWSNHSK (SEQ ID NO: 5); WCTQSWNNQAWNSPFYNCGEESK (SEQ ID NO: 6); WNTQTWCTQSWNNQAWNSPFYNCGEESK (SEQ ID NO: 7); WGSQTWTNPTWSSQTWTNPTWNNQTK (SEQ ID NO: 8); WTNPTWSSQAWTAQSWNGQPWNAAPK (SEQ ID NO: 9); WSNQTWNNSTWSNQTQNIQSWSNHSRRRKK (SEQ ID NO: 10); WSNQTWNNSTWSNQTRRRRRRKKC (SEQ ID NO: 11); WSNQTWNNSTWSNQTQNIQSWSNHSWNTQTWCTQSWNNQAWNSPFYN (SEQ ID NO: 12); WSNQTWNNSTWSNQTQNIQSWSNHSWNTQTWCTQSWNNQAWNSPFYNCGEESM (SEQ ID NO: 13); WGSQTWTNPTWSSQTWTNPTWNNQTKWTNPTWSSQAWTAQSWNGQPWNAAPK (SEQ ID NO: 14); derivatives thereof, analogs thereof, homologs thereof, and combinations thereof.

In some embodiments, the isolated peptides of the present disclosure include SEQ ID NO: 1. In some embodiments, the isolated peptides of the present disclosure include a sequence with at least 95% sequence identity to SEQ ID NO: 1. In some embodiments, the isolated peptides of the present disclosure include a sequence with at least 90% sequence identity to SEQ ID NO: 1. In some embodiments, the isolated peptides of the present disclosure include a sequence with at least 85% sequence identity to SEQ ID NO: 1. In some embodiments, the isolated peptides of the present disclosure include a sequence with at least 80% sequence identity to SEQ ID NO: 1. In some embodiments, the isolated peptides of the present disclosure include a sequence with at least 65% sequence identity to SEQ ID NO: 1.

In some embodiments, the isolated peptides of the present disclosure include SEQ ID NO: 2. In some embodiments, the isolated peptides of the present disclosure include a sequence with at least 95% sequence identity to SEQ ID NO: 2. In some embodiments, the isolated peptides of the present disclosure include a sequence with at least 90% sequence identity to SEQ ID NO: 2. In some embodiments, the isolated peptides of the present disclosure include a sequence with at least 85% sequence identity to SEQ ID NO: 2. In some embodiments, the isolated peptides of the present disclosure include a sequence with at least 80% sequence identity to SEQ ID NO: 2. In some embodiments, the isolated peptides of the present disclosure include a sequence with at least 65% sequence identity to SEQ ID NO: 2.

In some embodiments, the isolated peptides of the present disclosure include SEQ ID NO: 3. In some embodiments, the isolated peptides of the present disclosure include a sequence with at least 95% sequence identity to SEQ ID NO: 3. In some embodiments, the isolated peptides of the present disclosure include a sequence with at least 90% sequence identity to SEQ ID NO: 3. In some embodiments, the isolated peptides of the present disclosure include a sequence with at least 85% sequence identity to SEQ ID NO: 3. In some embodiments, the isolated peptides of the present disclosure include a sequence with at least 80% sequence identity to SEQ ID NO: 3. In some embodiments, the isolated peptides of the present disclosure include a sequence with at least 65% sequence identity to SEQ ID NO: 3.

In some embodiments, the isolated peptides of the present disclosure include SEQ ID NO: 4. In some embodiments, the isolated peptides of the present disclosure include a sequence with at least 95% sequence identity to SEQ ID NO: 4. In some embodiments, the isolated peptides of the present disclosure include a sequence with at least 90% sequence identity to SEQ ID NO: 4. In some embodiments, the isolated peptides of the present disclosure include a sequence with at least 85% sequence identity to SEQ ID NO: 4. In some embodiments, the isolated peptides of the present disclosure include a sequence with at least 80% sequence identity to SEQ ID NO: 4. In some embodiments, the isolated peptides of the present disclosure include a sequence with at least 65% sequence identity to SEQ ID NO: 4.

In some embodiments, the isolated peptides of the present disclosure include SEQ ID NO: 5. In some embodiments, the isolated peptides of the present disclosure include a sequence with at least 95% sequence identity to SEQ ID NO: 5. In some embodiments, the isolated peptides of the present disclosure include a sequence with at least 90% sequence identity to SEQ ID NO: 5. In some embodiments, the isolated peptides of the present disclosure include a sequence with at least 85% sequence identity to SEQ ID NO: 5. In some embodiments, the isolated peptides of the present disclosure include a sequence with at least 80% sequence identity to SEQ ID NO: 5. In some embodiments, the isolated peptides of the present disclosure include a sequence with at least 65% sequence identity to SEQ ID NO: 5.

In some embodiments, the isolated peptides of the present disclosure include SEQ ID NO: 6. In some embodiments, the isolated peptides of the present disclosure include a sequence with at least 95% sequence identity to SEQ ID NO: 6. In some embodiments, the isolated peptides of the present disclosure include a sequence with at least 90% sequence identity to SEQ ID NO: 6. In some embodiments, the isolated peptides of the present disclosure include a sequence with at least 85% sequence identity to SEQ ID NO: 6. In some embodiments, the isolated peptides of the present disclosure include a sequence with at least 80% sequence identity to SEQ ID NO: 6. In some embodiments, the isolated peptides of the present disclosure include a sequence with at least 65% sequence identity to SEQ ID NO: 6.

In some embodiments, the isolated peptides of the present disclosure include SEQ ID NO: 7. In some embodiments, the isolated peptides of the present disclosure include a sequence with at least 95% sequence identity to SEQ ID NO: 7. In some embodiments, the isolated peptides of the present disclosure include a sequence with at least 90% sequence identity to SEQ ID NO: 7. In some embodiments, the isolated peptides of the present disclosure include a sequence with at least 85% sequence identity to SEQ ID NO: 7. In some embodiments, the isolated peptides of the present disclosure include a sequence with at least 80% sequence identity to SEQ ID NO: 7. In some embodiments, the isolated peptides of the present disclosure include a sequence with at least 65% sequence identity to SEQ ID NO: 7.

In some embodiments, the isolated peptides of the present disclosure include SEQ ID NO: 8. In some embodiments, the isolated peptides of the present disclosure include a sequence with at least 95% sequence identity to SEQ ID NO: 8. In some embodiments, the isolated peptides of the present disclosure include a sequence with at least 90% sequence identity to SEQ ID NO: 8. In some embodiments, the isolated peptides of the present disclosure include a sequence with at least 85% sequence identity to SEQ ID NO: 8. In some embodiments, the isolated peptides of the present disclosure include a sequence with at least 80% sequence identity to SEQ ID NO: 8. In some embodiments, the isolated peptides of the present disclosure include a sequence with at least 65% sequence identity to SEQ ID NO: 8.

In some embodiments, the isolated peptides of the present disclosure include SEQ ID NO: 9. In some embodiments, the isolated peptides of the present disclosure include a sequence with at least 95% sequence identity to SEQ ID NO: 9. In some embodiments, the isolated peptides of the present disclosure include a sequence with at least 90% sequence identity to SEQ ID NO: 9. In some embodiments, the isolated peptides of the present disclosure include a sequence with at least 85% sequence identity to SEQ ID NO: 9. In some embodiments, the isolated peptides of the present disclosure include a sequence with at least 80% sequence identity to SEQ ID NO: 9. In some embodiments, the isolated peptides of the present disclosure include a sequence with at least 65% sequence identity to SEQ ID NO: 9.

In some embodiments, the isolated peptides of the present disclosure include SEQ ID NO: 10. In some embodiments, the isolated peptides of the present disclosure include a sequence with at least 95% sequence identity to SEQ ID NO: 10. In some embodiments, the isolated peptides of the present disclosure include a sequence with at least 90% sequence identity to SEQ ID NO: 10. In some embodiments, the isolated peptides of the present disclosure include a sequence with at least 85% sequence identity to SEQ ID NO: 10. In some embodiments, the isolated peptides of the present disclosure include a sequence with at least 80% sequence identity to SEQ ID NO: 10. In some embodiments, the isolated peptides of the present disclosure include a sequence with at least 65% sequence identity to SEQ ID NO: 10

In some embodiments, the isolated peptides of the present disclosure include SEQ ID NO: 11. In some embodiments, the isolated peptides of the present disclosure include a sequence with at least 95% sequence identity to SEQ ID NO: 11. In some embodiments, the isolated peptides of the present disclosure include a sequence with at least 90% sequence identity to SEQ ID NO: 11. In some embodiments, the isolated peptides of the present disclosure include a sequence with at least 85% sequence identity to SEQ ID NO: 11. In some embodiments, the isolated peptides of the present disclosure include a sequence with at least 80% sequence identity to SEQ ID NO: 11. In some embodiments, the isolated peptides of the present disclosure include a sequence with at least 65% sequence identity to SEQ ID NO: 11.

In some embodiments, the isolated peptides of the present disclosure include SEQ ID NO: 12. In some embodiments, the isolated peptides of the present disclosure include a sequence with at least 95% sequence identity to SEQ ID NO: 12. In some embodiments, the isolated peptides of the present disclosure include a sequence with at least 90% sequence identity to SEQ ID NO: 12. In some embodiments, the isolated peptides of the present disclosure include a sequence with at least 85% sequence identity to SEQ ID NO: 12. In some embodiments, the isolated peptides of the present disclosure include a sequence with at least 80% sequence identity to SEQ ID NO: 12. In some embodiments, the isolated peptides of the present disclosure include a sequence with at least 65% sequence identity to SEQ ID NO: 12.

In some embodiments, the isolated peptides of the present disclosure include SEQ ID NO: 13. In some embodiments, the isolated peptides of the present disclosure include a sequence with at least 95% sequence identity to SEQ ID NO: 13. In some embodiments, the isolated peptides of the present disclosure include a sequence with at least 90% sequence identity to SEQ ID NO: 13. In some embodiments, the isolated peptides of the present disclosure include a sequence with at least 85% sequence identity to SEQ ID NO: 13. In some embodiments, the isolated peptides of the present disclosure include a sequence with at least 80% sequence identity to SEQ ID NO: 13. In some embodiments, the isolated peptides of the present disclosure include a sequence with at least 65% sequence identity to SEQ ID NO: 13.

In some embodiments, the isolated peptides of the present disclosure include SEQ ID NO: 14. In some embodiments, the isolated peptides of the present disclosure include a sequence with at least 95% sequence identity to SEQ ID NO: 14. In some embodiments, the isolated peptides of the present disclosure include a sequence with at least 90% sequence identity to SEQ ID NO: 14. In some embodiments, the isolated peptides of the present disclosure include a sequence with at least 85% sequence identity to SEQ ID NO: 14. In some embodiments, the isolated peptides of the present disclosure include a sequence with at least 80% sequence identity to SEQ ID NO: 14. In some embodiments, the isolated peptides of the present disclosure include a sequence with at least 65% sequence identity to SEQ ID NO: 14.

### Analogs

In some embodiments, the isolated peptides of the present disclosure include an analog of any one of SEQ ID NOS: 1-14. In some embodiments, the analog is at least 95% identical to any of SEQ ID NOS: 1-14. In some embodiments, the analog is at least 90% identical to any of SEQ ID NOS: 1-14. In some embodiments, the analog is at least 85% identical to any of SEQ ID NOS: 1-14. In some embodiments, the analog is at least 80% identical to any of SEQ ID NOS: 1-14. In some embodiments, the analog is at least 65% identical to any of SEQ ID NOS: 1-14.

### Homologs

In some embodiments, the isolated peptides of the present disclosure include a homolog of any one of SEQ ID NOS: 1-14. In some embodiments, the homolog is at least 95% identical to any of SEQ ID NOS: 1-14. In some embodiments, the homolog is at least 90% identical to any of SEQ ID NOS: 1-14. In some embodiments, the homolog is at least 85% identical to any of SEQ ID NOS: 1-14. In some embodiments, the homolog is at least 80% identical to any of SEQ ID NOS: 1-14. In some embodiments, the homolog is at least 65% identical to any of SEQ ID NOS: 1-14.

### Derivatives

In some embodiments, the isolated peptides of the present disclosure include a derivative of any one of SEQ ID NOS: 1-14. In some embodiments, the derivative includes one or more amino acid moieties derivatized with one or more functional groups. In some embodiments, the one or more functional groups are positioned on amino acid backbones, R groups, or combinations thereof. In some embodiments, the one or more functional groups can include, without limitation, alkanes, alkenes, ethers, alkynes, alkoxyls, aldehydes, carboxyls, hydroxyls, hydrogens, sulfurs, phenyls, cyclic rings, aromatic rings, heterocyclic rings, linkers, and combinations thereof.

In some embodiments, the isolated peptides of the present disclosure include a single isolated peptide. In some embodiments, the isolated peptides of the present disclosure include a plurality of isolated peptides. In some embodiments, each of the plurality of isolated peptides includes a sequence of any one of SEQ ID NOS: 1-14, derivatives thereof, analogs thereof, homologs thereof, and combinations thereof.

### Origins

The isolated peptides of the present disclosure can be derived from various NANOGs. For example, in some embodiments, the NANOG is derived from one of humans, non-human mammals, mice, chimpanzees, dogs, cats, chickens, or zebrafish. In some embodiments, the NANOG is derived from a human.

### Applications

The isolated peptides of the present disclosure can have numerous applications. For example, in some embodiments, the isolated peptides of the present disclosure are suitable for use in an application that can include, without limitation, chromatin reorganization, conversion of cells to pluripotent stem cells, cellular growth, delivery of materials into cells, utilization as tissue adhesives, utilization as hydrogel scaffolds, utilization as components in topical creams, utilization as immobilizing agents of various materials, utilization as scaffolds for cellular growth, utilization as diagnostics or therapies, and combinations thereof.

### Delivery of Peptides Into Cells

Further embodiments of the present disclosure pertain to methods of delivering the isolated peptides of the present disclosure into cells. Such methods generally include exposing the cells to the isolated peptides of the present disclosure, nucleotide sequences that express the isolated peptides of the present disclosure, or combinations thereof.

In some embodiments, the exposing includes exposing the cells to the isolated peptides of the present disclosure to result in the delivery of the isolated peptide into the cells. In some embodiments, the exposing includes exposing the cells to a nucleotide sequence that expresses the isolated peptides of the present disclosure to result in the expression of the isolated peptides in the cells.

The methods of the present disclosure can have numerous embodiments. For example, the methods of the present disclosure can be utilized to deliver various isolated peptides into cells through various routes.

### Direct Delivery of Isolated Peptides Into Cells

In some embodiments, the methods of the present disclosure include the direct delivery of the isolated peptides of the present disclosure into cells. In some embodiments, the isolated peptides of the present disclosure may be delivered into cells without any additional materials. In some embodiments, the isolated peptides of the present disclosure may be delivered into cells with additional materials. For instance, in some embodiments, the additional materials include, without limitation, small molecules, drugs, proteins, enzymes, catalysts, virus particles, nucleotides, DNA, RNA, plasmids, and combinations thereof. In some embodiments, the additional materials may be embedded with the isolated peptides of the present disclosure.

### Expression of Isolated Peptides in Cells

In some embodiments, the methods of the present disclosure include the expression of the isolated peptides of the present disclosure in cells. In some embodiments, the expression occurs by exposing the cells to a nucleotide sequence that expresses the isolated peptides of the present disclosure. In some embodiments, the nucleotide sequence includes, without limitation, DNA, cDNA, RNA, mRNA, or combinations thereof.

In some embodiments, the nucleotide sequence may also include a promoter that is operably linked to the nucleotide sequence for expressing the nucleotide sequence. In some embodiments, the nucleotide sequence may be part of an expression vector, such as a plasmid.

### Cells

The isolated peptides and nucleotide sequences of the present disclosure may be exposed to various types of cells. For instance, in some embodiments, the cells include human cells. In some embodiments, the cells include animal cells, such as murine cells.

In some embodiments, the cells include stem cells. In some embodiments, the exposing results in the conversion of the cells to pluripotent stem cells.

### Exposing

Various methods may be utilized to expose cells to the isolated peptides and nucleotide sequences of the present disclosure. For instance, in some embodiments, the exposing occurs in vitro. In some embodiments, the exposing includes incubating the cells with the isolated peptides and nucleotide sequences of the present disclosure.

In some embodiments, the exposing occurs in vivo in a subject. In some embodiments, the exposing includes administering the isolated peptides and nucleotide sequences of the present disclosure to the subject. In some embodiments, the administering occurs by a method that can include, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, and combinations thereof.

In some embodiments, the isolated peptides and nucleotide sequences of the present disclosure is used to treat or prevent a disorder or disease in the subject. In some embodiments, the isolated peptides and nucleotide sequences of the present disclosure may be in the form of a drug that specifically treats or prevents a particular disease, such as cancer, a neurodegenerative disease, or combinations thereof.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1

### NANOG Prion-Like Assembly Mediates DNA Bridging

NANOG controls entry to stem cell pluripotency. In particular, human NANOG expression is necessary to reset human stem cells to pluripotent ground state with unlimited self-renewal potential similar to mouse embryonic stem cells (ESC). In this Example, Applicant characterized human NANOG and identified unique features that relate to its dose-sensitive function as a master and pioneering transcription factor.

NANOG is largely disordered with an N-terminal unstructured region and a C-terminal prion-like domain (PrD). NANOG PrD easily aggregates and phase transitions to gel-like condensates. Using high-sensitivity fluorescence techniques, Applicant demonstrates in this Example that full-length NANOG readily forms higher order oligomers at low nM concentrations, orders of magnitude lower than typical for amyloid or supramolecular assemblies. Using single molecule FRET and cross-correlation techniques, Applicant demonstrates that NANOG oligomerization is essential for bridging DNA elements together.

Additionally, Applicant's results provide a physical basis for NANOG's indispensable role in shaping the pluripotent genome. Without being bound by theory, Applicant proposes that NANOG's unique ability to form prion-like assemblies provides a cooperative and concerted DNA bridging mechanism essential for chromatin reorganization and dose-sensitive activation of ground state pluripotency.

NANOG gates access to unrestricted self-regeneration and germline development. NANOG levels are tightly regulated in cells—high levels correlate with reprogramming and self-renewal while low levels lead to spontaneous differentiation. This striking dose sensitivity is linked to Nanog gene's monoallelic to biallelic expression switch as the cell transitions towards ground state pluripotency. Furthermore, a landmark study showed that human stem cells in vitro could achieve ground state pluripotency similar to mouse ESCs simply by inducing additional NANOG expression.

The pluripotent genome is shaped by master transcription factors NANOG and OCT4. Recent studies have proposed that chromatin reorganization could also occur through the physical mechanism of liquid-liquid phase separation (LLPS). Recently, Applicant showed that KLF4 undergoes LLPS, facilitating its key role in cellular reprogramming. LLPS is greatly facilitated by intrinsically disordered prion-like domains (PrD) present in many RNA-and DNA-binding proteins. Thus, Applicant set to characterize what unique features in NANOG that can explain its key role in activating pluripotency.

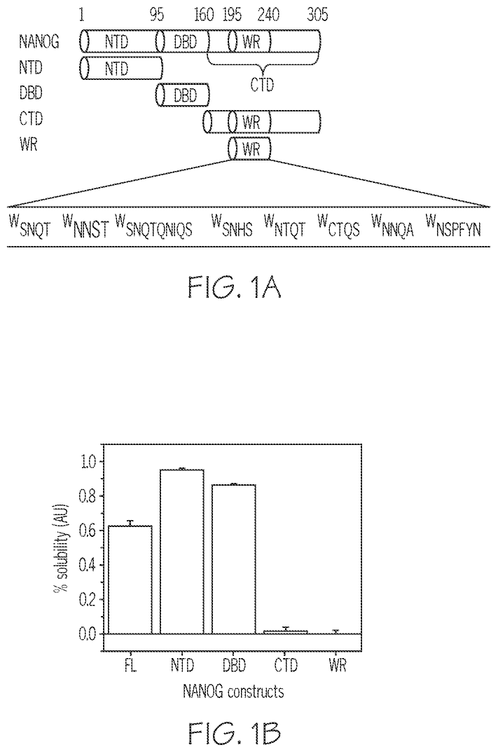

Early studies pinpoint NANOG's ability to dimerize. Self-association is essential for NANOG pluripotency function regardless of species origin and sequence conservation. Previous studies reported that mouse NANOG dimerizes (˜3 μM concentration) through its tryptophan-repeat (WR) domain (FIG. 1A). By pull-down experiments, human NANOG was also reported to self-associate via its WR domain.

Human NANOG is aggregation-prone and it has limited biophysical characterization of the protein and its subdomains, except for its DNA-binding domain (DBD). To delineate regions that contribute to aggregation, Applicant determined each domain's relative solubility. NANOG NTD and DBD were highly soluble, while WR-containing CTD constructs have limited solubility (FIG. 1B).

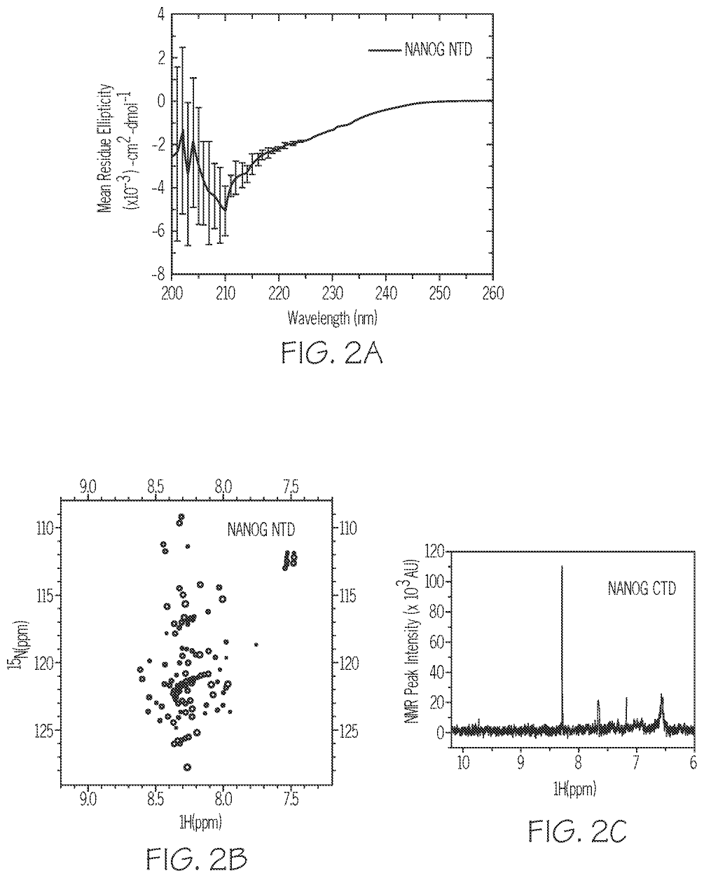

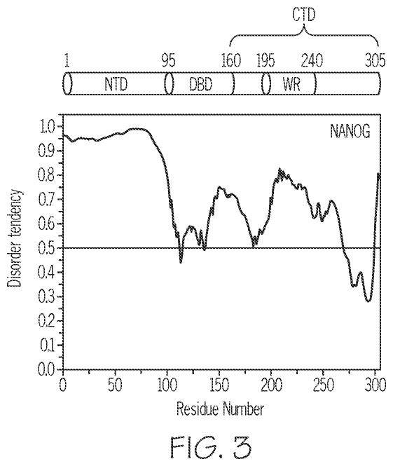

Applicant further observed that NANOG NTD was intrinsically disordered, exhibiting random coil CD spectral signature and narrow ¹H peak dispersion in 2D ¹⁵N HSQC NMR spectra (FIGS. 2A-2C), consistent with NANOG computational disorder prediction. NANOG DBD has been well-characterized in the literature as a folded domain with nM-μM DNA binding affinity. In stark contrast to NTD and DBD, NANOG CTD is highly aggregation-prone. Even at concentrations as low as 10 μM, signals in a 1D ¹H-NMR spectra were hardly detectable (FIG. 3).

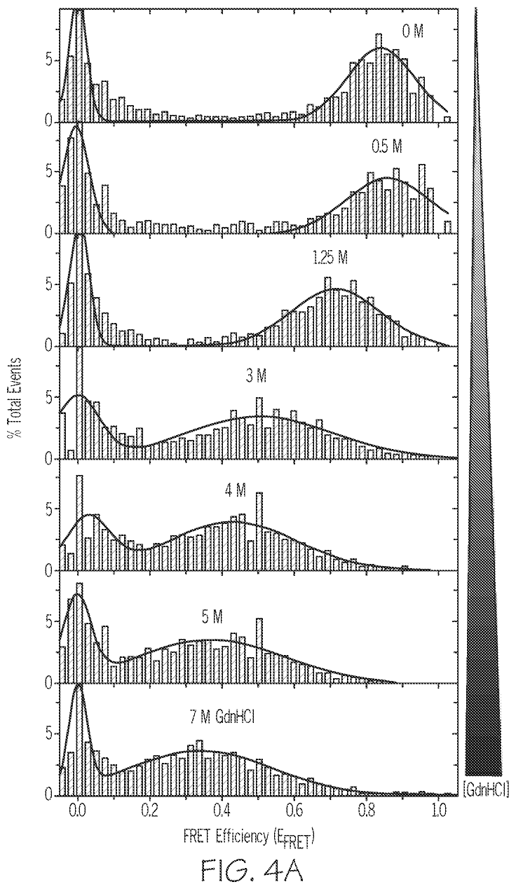

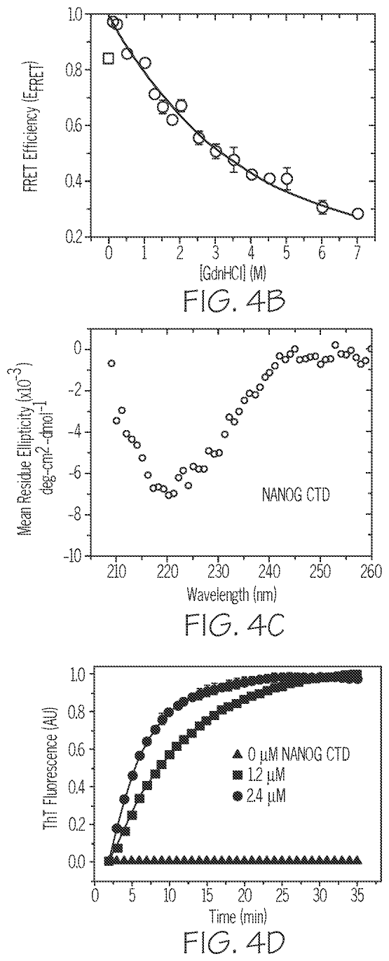

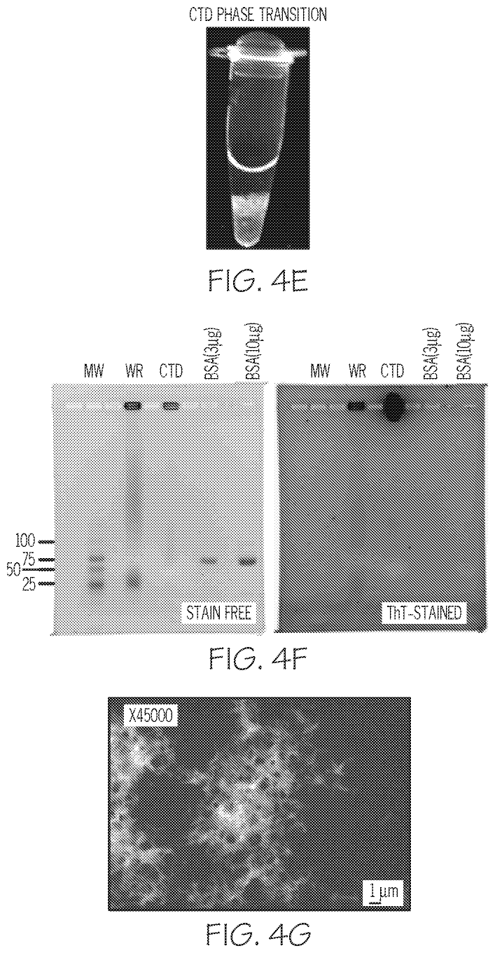

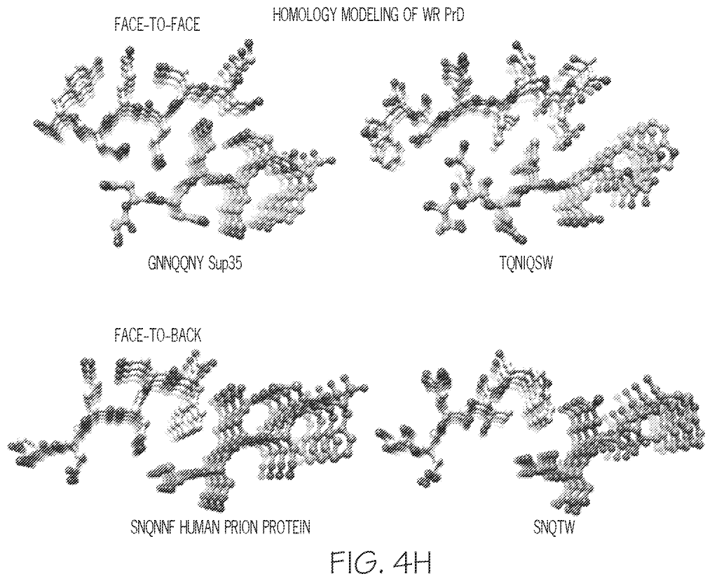

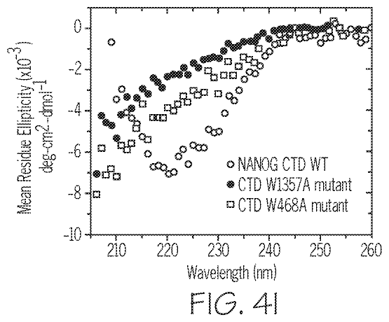

To probe the NANOG CTD structure, Applicant utilized smFRET (single-molecule Förster/fluorescence resonance energy transfer), a technique ideal for aggregation-prone systems where experiments are performed in dilute picomolar concentrations. NANOG CTD was mutated to introduce two Cys residues at positions 183 and 243, flanking the WR domain. Standard guanidinium hydrochloride (GndHCl) protein denaturation experiments were performed using NANOG CTD doubly fluorescently labeled with Alexa Fluor 488 and Alexa Fluor 594 (AF488/AF594). Applicant observed shifts in FRET efficiencies towards lower values as expected for protein expansion associated with denaturation (FIG. 4A). However, the changes in NANOG CTD FRET followed a non-cooperative transition (FIG. 4B), which indicated that CTD did not have a persistent structure or lacked a strong compact, hydrophobic core.

However, Applicant acquired CD spectra at 2 μM protein concentration and observed β-sheet secondary structure (FIG. 4C), indicating that CTD can undergo disorder-to-order transition. Applicant speculated that the structural transition is linked to amyloid formation since the WR domain sequence is strikingly similar to amyloid prions or prion-like domains (FIG. 4A), which are rich in polar residues (Ser, Asn, and Gln) and hydrophobic residues. Indeed, ThT aggregation assays showed rapid aggregation and the kinetics was dependent on protein concentration (FIG. 4D). At low μM concentrations in the absence of crowding agents, NANOG CTD easily phase transitions to gel-like condensates (FIG. 4E) that were ThT-positive and migrated as high MW complexes in SDD-AGE (semi-denaturing detergent agarose gel electrophoresis) (FIG. 4F).

Scanning electron micrographs of NANOG CTD gel revealed networks of fibril-like structures (FIG. 4G). To investigate whether Trp residues play a major role in aggregation, Applicant mutated three (W468A) or four (W1357A) alternating Trp residues in the WR repeat sequence to Ala. This resulted in a reduction of β-sheet structures to more random coil-like (FIG. 4H).

Solubility issues of the WR domain or peptides prevented experimental high-resolution structural studies. Alternatively, Applicant performed computational modeling of two WR repeat sequences most homologous to published x-ray structures of peptide prions: yeast Sup35 and human prion protein (FIG. 4H). Both WR structures showed steric zipping of β-strands but were modeled in different orientations suggesting that WR domain does not adopt a unique structure. Heterogeneous orientations of WR assembly may allow spatial flexibility for NANOG domains to interact with DNA and other proteins.

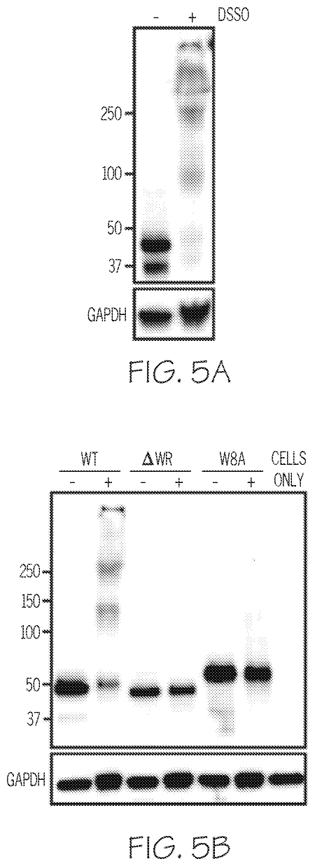

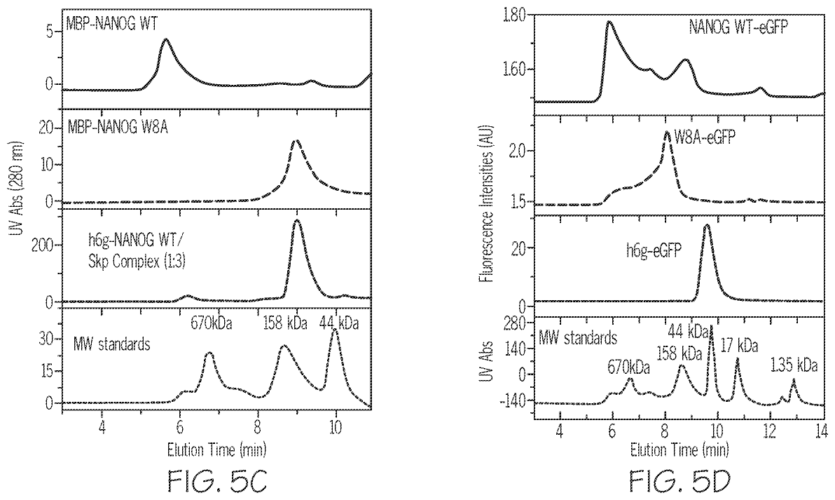

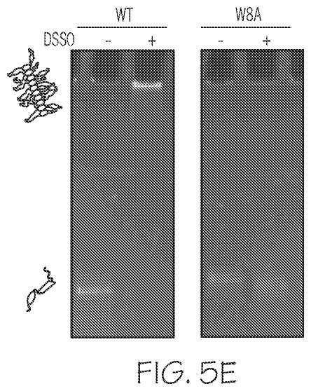

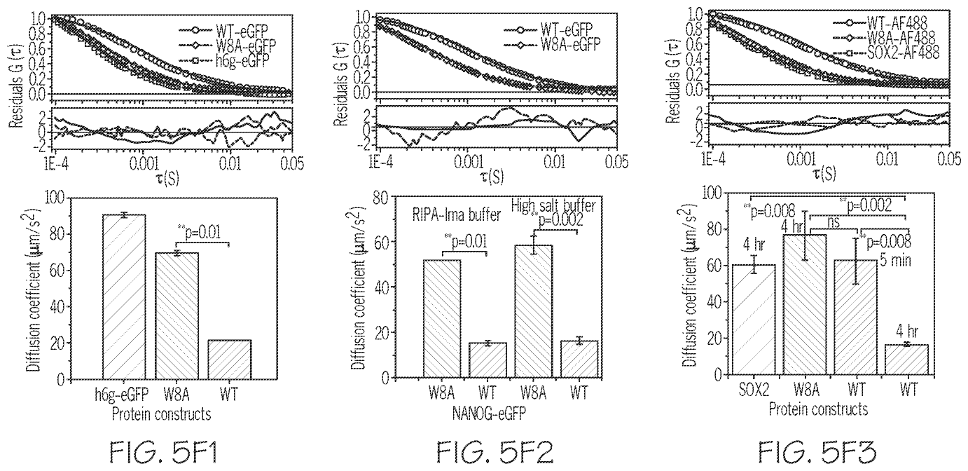

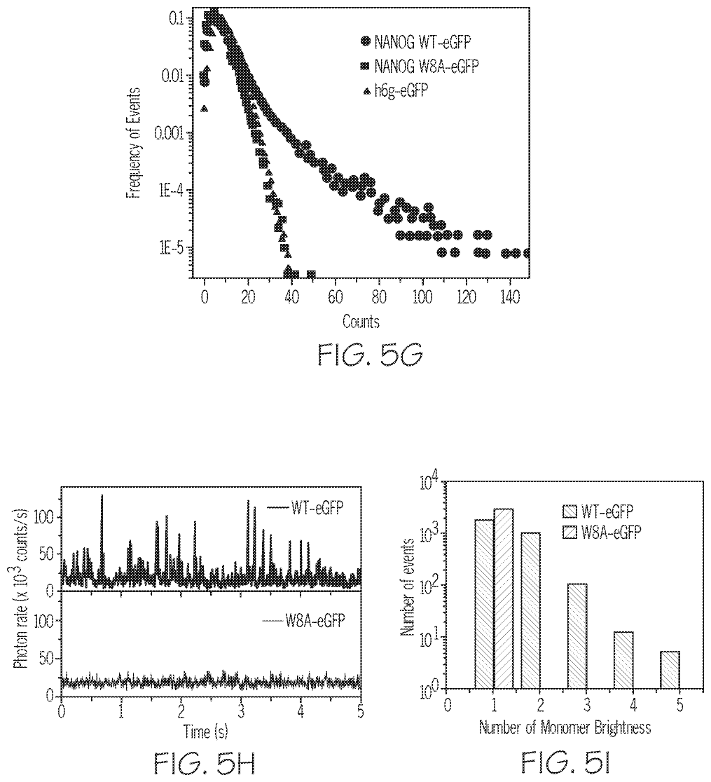

Applicant next investigated if oligomerization translates to the full-length protein NANOG. Applicant tested if endogenous NANOG could spontaneously assemble in H9 human embryonic stem cells (ESCs) as well as nucleofected NANOG in differentiated HEK 293T mammalian cells. In the presence of DSSO chemical crosslinker, NANOG readily crosslinked to form dimers and other high MW complexes in both cell types (FIGS. 5A-B). To check the role of the WR domain in oligomerization, Applicant generated WR mutants (ΔWR deletion and W8A mutant where all eight Trp residues were mutated to Ala) and transfected them into HEK 293T cells. Consistently, Applicant observed that WT NANOG easily crosslinked to higher order species while the WR mutants could not.

Since NANOG is a known hub protein that interacts with many cellular proteins, oligomerization in cells may not be solely due to NANOG self-assembly. Thus, Applicant characterized oligomerization behavior with purified full-length NANOG. Applicant prepared NANOG constructs with GB1-and MBP-fused tags to increase solubility. Applicant also generated NANOG W8A mutant with all eight WR-Trp residues mutated to Ala for side-by-side evaluation. GB1 fusion resulted in the accumulation of WT protein inside inclusion bodies upon E. coli expression. However, consistent with previous observations, GB1-fusion NANOG WT became soluble when co-expressed with the Skp chaperone. Using size exclusion chromatography (SEC), both Skp and GB1-fused NANOG WT proteins co-eluted as a 3:1 complex (FIG. 5C).

Without being bound by theory, Applicant envisions that the trimeric Skp chaperone provides a hydrophobic cage that wraps around NANOG WT and interacts with exposed Trp residues. This is consistent with the co-elution of WT but not the W8A mutant. With MBP fusions, the proteins were soluble.

However, strong detergents were necessary for the purification process. To verify that purified proteins were still functional for DNA binding, fluorescence-based electrophoretic mobility shift assays (fEMSA) assays were performed. Despite binding DNA effectively, MBP-NANOG WT eluted as a high MW aggregate by SEC (FIG. 5C). In contrast, the W8A mutant migrated as a monomeric peak based on estimated MW (FIG. 5C).

Applicant also purified GFP-tagged NANOG from HEK 293T cells, speculating that low expression makes it less prone to aggregation. Interestingly, using fluorescence-detected SEC and 10 nM injected concentrations, GFP-tagged WT eluted in the void volume (high MW complex>2 MDa) and W8A migrated mostly as a monomeric peak (FIG. 5D). DSSO crosslinking of purified proteins confirmed that WT assembled readily, immobilized in SDS-PAGE wells, while W8A failed to crosslink intermolecularly (FIG. 5E).

Next, Applicant characterized NANOG oligomerization states at low nM concentrations using fluorescence fluctuations spectroscopy (FFS). By quantifying the fluorescence intensities of a few molecules at a time, FFS data can be analyzed using different strategies: Fluorescence Correlation Spectroscopy (FCS) to obtain the molecule's diffusion coefficient, PCH (Photon counting histogram), and burst analysis to access the molecule's ‘brightness’ distribution. The brightness parameter provides insight to the oligomer size. For instance, a dimer will have twice the brightness of a monomer.

With FCS, Applicant investigated NANOG diffusion behavior using different NANOG preparations and solution conditions: (i) GFP-tagged in mammalian cell lysates; (ii) purified GFP-tagged in vitro with detergent and high salt buffer; and (iii) purified AF488-conjugated NANOG in vitro. To estimate the molecular sizes, Applicant used the empirical equations relating hydrodynamic radii and the number of residues for folded and denatured proteins. Both the positive control hog-eGFP and GFP-tagged W8A mutant had diffusion coefficients consistent with their monomeric sizes, falling within the molecular size boundary calculations for folded and denatured proteins (FIGS. 5F1-3). Meanwhile, GFP-tagged NANOG WT (in vitro and in mammalian cell lysates) diffused three times slower than W8A mutant, and its hydrodynamic radii (Rh) were significantly outside the monomeric size range even with the assumption that NANOG is completely disordered.

Consistently, data with AF488-conjugated, refolded proteins (FIGS. 5F1-3) show that SOX2-AF488 (similar size disordered protein) and mutant W8A-AF488 were in good agreement with monomeric sizes while WT-AF488 behaved more like a large oligomer. Back-calculation from the diffusion coefficients to the number of residues using the empirical equations estimated that WT could be 8-100× larger than the W8A mutant.

Using independent PCH analysis, hog-eGFP and GFP-tagged W8A show photon count distributions that could be approximated by a Poisson distribution of a particle with uniform molecular brightness (FIG. 5G). In contrast, WT revealed a ‘long tail’ distribution, indicative of large aggregates. Consequently, its PCH curve could only be fitted segmentally, each fit characterized by a different molecular brightness or oligomer size.

Alternatively, using burst analysis or direct observation of raw intensity fluctuations, Applicant observed that W8A mutant had even or uniform fluorescence intensity fluctuations while WT had ‘bursts’ or large intensity deviations (˜10-12× from the average fluctuations; FIG. 5H). To estimate the sizes of the oligomers present, Applicant assumed that the average intensity fluctuations represented the monomeric species and the data were binned based on average counts per second (cps).

Consistent with other data, W8A mutant showed only monomeric distribution (FIG. 5I). WT, however, showed several oligomeric size distributions and not a distinct oligomeric size. Applicant noted similar trends in FFS experiments of mCherry-tagged NANOG in cell lysates. More oligomers were observed in the presence of WR PrD (NANOG-mCherry) than in the absence (NANOG ΔWR-mCherry). Thus, NANOG could self-assemble readily at low nM concentrations.

To further validate that NANOG cellular concentration permits its self-assembly, Applicant estimated endogenous NANOG cellular concentration in H9 ESCs using GFP (with h6-eGFP and GFP-tagged NANOG) calibrations and western blot imaging. Both endogenous NANOG in H9 ESCs and GFP-tagged NANOG expressed in HEK 293T cells were found to be ˜70-80 nM, well above the ˜5 nM concentrations used in FFS experiments. Thus, without being bound by theory, Applicant expects NANOG to self-assemble into higher-order oligomers once expressed in cells.

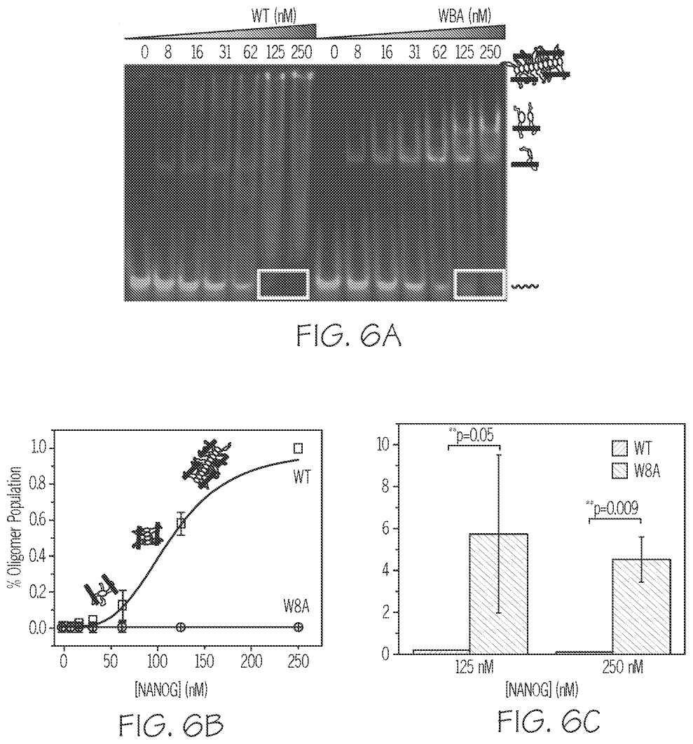

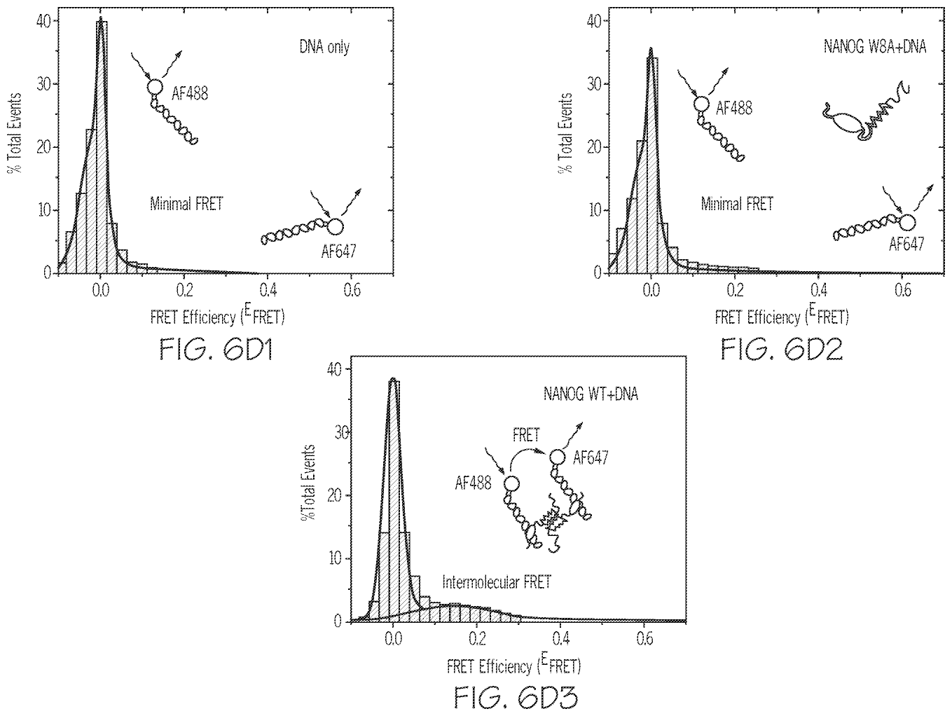

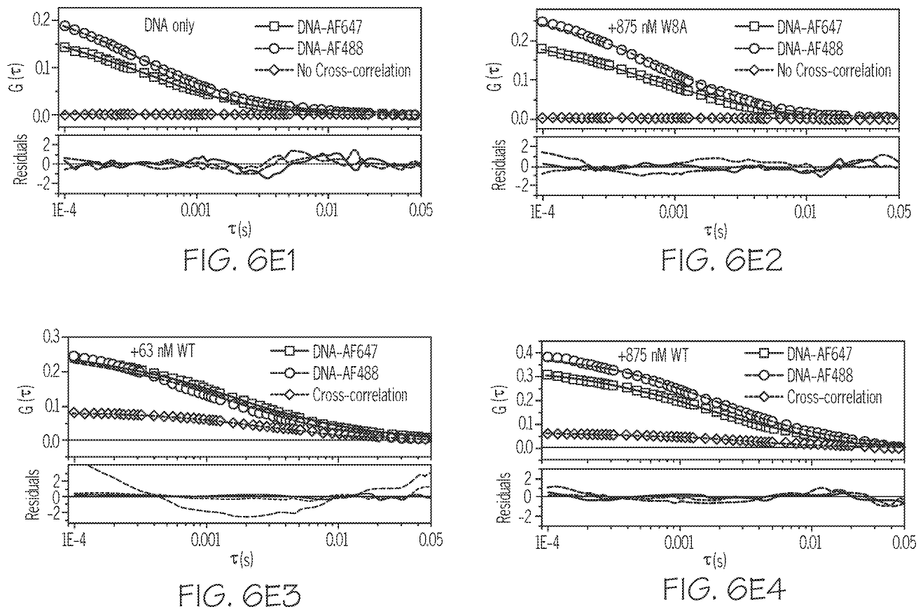

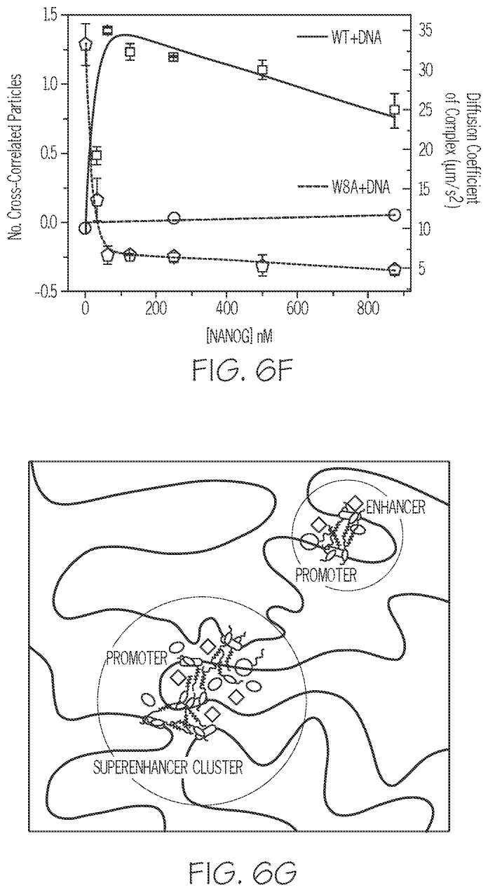

Applicant speculates that NANOG assembly properties have relevance to its function as a master TF. To investigate how oligomerization affects DNA binding, Applicant performed fEMSA with untagged WT and W8A mutants (FIG. 6A), as well as GFP-tagged WT and W8A. Both NANOG WT and W8A bound DNA effectively due to the presence of DBD. However, Applicant observed dramatic differences in band and migration patterns. There were two distinct bands with the W8A mutant, representing singly and doubly bound to the Gata6 DNA (40 bp with two cognate sites). However, with WT. Applicant observed one distinct band (singly-bound) and a smeared distribution consistent with variably sized oligomers. At higher NANOG concentrations, proteins were immobilized and remained inside the fEMSA gel wells, consistent with high MW DNA: NANOG complexes. Quantification of bands as a function of NANOG concentration revealed a sigmoidal curve consistent with a cooperative assembly process present in WT but not in W8A mutant (FIG. 6B).

Moreover, Applicant could observe a higher amount of DNA bound to WT than W8A mutant when larger NANOG oligomers are present (white rectangles in FIG. 6A; quantification in FIG. 6C). Consistently, fEMSA experiments of GFP-tagged NANOG with 40 bp Gata6 and other known DNA targets (257 bp p) Sat satellite DNA and 404 bp Nanog promoter) confirmed that WT bound DNA more cooperatively and tightly than the W8A mutant. The presence of DNA: NANOG oligomers suggests that NANOG might be able to bridge two isolated DNA through its CTD PrDs.

To test the DNA bridging hypothesis, Applicant performed single molecule FRET (smFRET) diffusion experiments. Intermolecular diffusion smFRET is challenging due to low chance encounters at dilute pM concentrations. Gata6 DNAs were independently labeled with AF488 or AF647. With DNA alone or in the presence of NANOG W8A, Applicant hardly observed any FRET efficiency (FIGS. 6D1-3). However, in the presence of 250 nM NANOG WT. Applicant observed FRET efficiency ˜0.15, which proved that WT was able to bring separate DNAs to within ˜70 Å proximity (FIGS. 6D1-3).

To further validate NANOG-dependent DNA bridging, Applicant resorted to fluorescence cross-correlation technique (FCCS). Binding of individual DNAs (differentially labeled with AF488 and AF647) to form a complex would result in correlated fluorescence signals. With the DNAs alone or with W8A mutant (FIGS. 6D1-3), no cross-correlation (blue line) was observed. However, in the presence of WT NANOG, Applicant observed significant cross-correlation (FIGS. 6E1-4).

FCCS experiments with various NANOG concentrations confirmed cross-correlation with as little as ˜30 nM WT NANOG (FIG. 6F). The increase in cross-correlated particles coincided with a decrease in molecular complex diffusion coefficient (bright lower line, FIG. 6F). Without being bound by theory, the decrease in cross-correlated particles at high NANOG concentrations may reflect competition for DNA with excess NANOG.

In summary, Applicant's data in this Example show that NANOG oligomerizes at low nM concentrations, at least three orders magnitude lower than most protein assemblies (amyloids, signalosomes, multivalent complexes). This unique property may explain NANOG's dose-sensitive action and why NANOG levels correlate with activation of pluripotency. As a pioneering transcription factor (TF), NANOG associates with high-density TF/coactivator superenhancer clusters and interacts with satellite DNA to decompact or remodel heterochromatin for the acquisition of pluripotency.

Applicant's results suggest how NANOG can mechanistically help shape the pluripotent genome (FIG. 6G). Applicant's data also corroborate 4C-seq studies where it was shown that extensive contacts mediated by the Nanog gene with other pluripotency-specific genes including intrachromosomal (e.g., Rybp, Ezh2, Tcf3, and Smarcad1) and interchromosomal contact genes such as Mybl2, Dppa5, Rex1, Zfp281, Lefty1, Lin28a, Esrrb, Klf5, Sall1, Cbx5 and Cbx7. Further, NANOG knockout resulted in reduced contact frequencies at clusters where pluripotency factors bind, and more importantly, they demonstrated that NANOG has a direct role in bringing distant loci together.

Introduction of synthetic NANOG that could target a specific locus led to newfound specific Nanog contacts with other pluripotency (Sall1 and Klf2) and developmental (Irx cluster) genes. Bridging of multiple intergenomic loci in a concerted manner can be readily accomplished with a prion-like assembly mechanism (FIG. 6G). NANOG prion-like assembly may also serve to assemble other coactivators/TFs through interaction with the PrD domain or other regions. Further studies with coactivators/TFs are necessary to assess homo-/hetero-oligomerization states and stoichiometric ratios. Nevertheless, NANOG's observed dose sensitivity strongly support Applicant's experimental observations.

The aggregation process is highly concentration-dependent. Therefore, without being bound by theory, Applicant proposes that the rise in NANOG levels triggers a timely switch, resulting in cooperative NANOG assembly in synchrony with chromatin reorganization required for activation of stem cell pluripotency.

### Example 2

### Novel Nanomaterials From NANOG Prion-Like Repeats

In this Example, Applicant identifies minimalistic regions within the multivalent WR domain of NANOG and properties that facilitate fibrillation and gelation. Linking additional basic residues generates mini-peptide constructs that mimic the full-length NANOG's cooperative ability to bridge DNA molecules. Modulating amino acid composition by focusing on the ‘hardening’ WR repeats and ‘softening’ charged residues enabled tuning for material and functional properties. NANOG WR-based peptides enable partitioning of biologics, and formation of nano-to macro-scale assemblies and 3D hydrogel scaffolds. NANOG bio-inspired materials may provide novel nanomaterials for translational applications in nanotechnology and therapeutics.

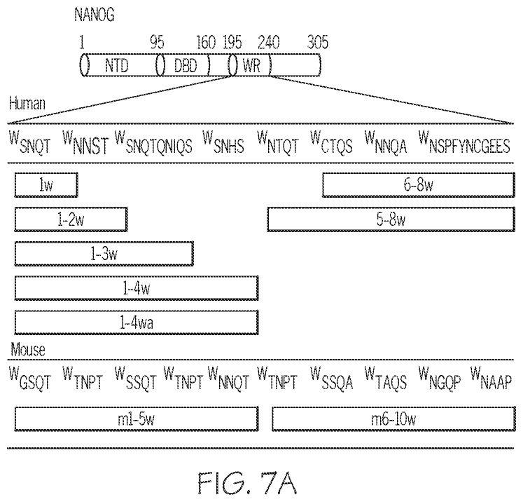

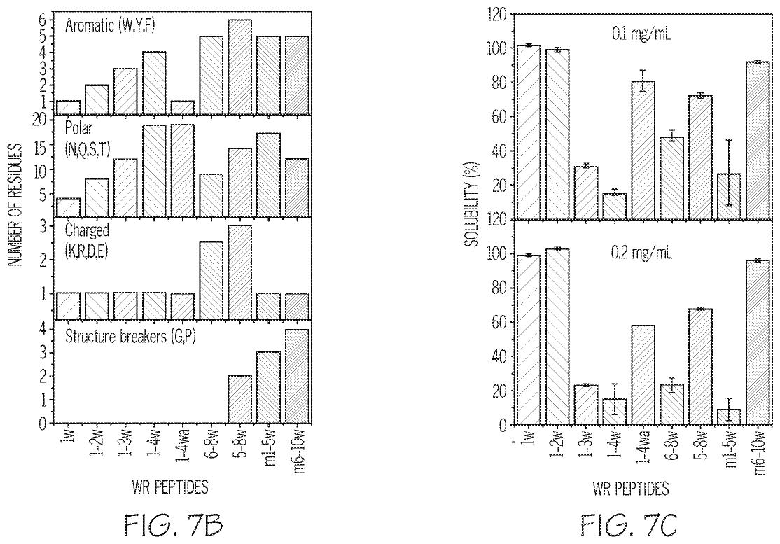

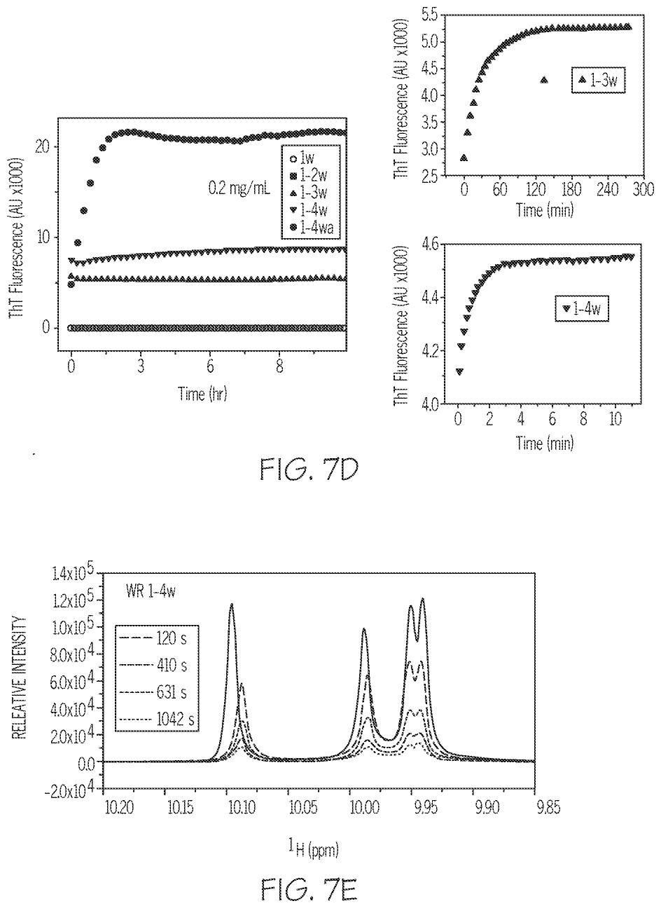

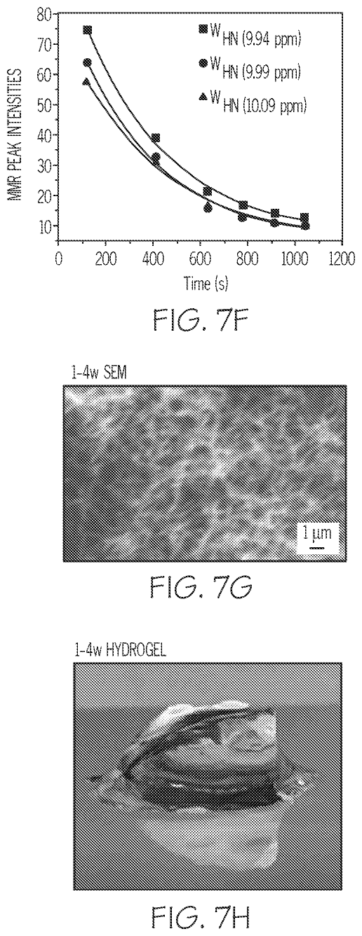

Applicant previously demonstrated that the full-length human NANOG behaves as a functional amyloid (Example 1). At low nM concentrations, NANOG readily self-assembles via its WR prion-like domain (PrD) (FIG. 7A) into large oligomers. The assembly process enables NANOG to function as a master transcription factor and efficiently recruit DNA. The WR PrD domain, composed of eight Trp pseudo repeats, is highly aggregation-prone. Applicant dissected and studied segments within the WR domain (FIGS. 7A-H) that contribute to aggregation propensity. Peptides containing both the N-terminal and C-terminal repeats were custom-synthesized (FIG. 7A). The mouse WR domain, which is known to self-dimerize and is comprised of 10 WR repeats, was also studied for comparison.

Amino acids of the synthesized peptides were classified into aromatic, polar, charged and structure breakers (FIG. 7B). Applicant observed a striking degree of correlation between amino acid type distribution, and the peptide's solubilities and aggregation propensities, as monitored by Thioflavin T (ThT, amyloid binding dye) aggregation kinetics assay (FIGS. 7B-7D). Formation of β-sheet fibrils were confirmed by CD spectroscopy and Transmission Electron Microscopy. Hydrophobic/aromatic residues as well as polar residues such as Gln and Asn are commonly found in amyloid or prion-like domains. On the other hand, charged and ‘structure breaker’ residues such as Gly and Pro contribute to the peptide's higher solubility. Increasing the number of WR repeats (i.e., comparing 1w through 1-4w) resulted in lower solubility and higher ThT fluorescence. While 1w and 1-2w peptides were very soluble at 0.1 and 0.2 mg/mL exhibiting minimal amyloid-positive ThT fluorescence, both 1-3w and 1-4w were more insoluble and positive for amyloid (FIGS. 7B-D). Aggregation kinetics was rapid; 1-4w reached maximal ThT fluorescence within 2-3 min and 1-3w ˜20 min (FIG. 7D).

NMR spectroscopy showed rapid peak broadening for tryptophan side chains within minutes, suggesting intermolecular interactions (FIGS. 7E-F). Consequently, the 1-4w peptide readily formed 3D fibril-like networks as imaged by scanning electron microscopy (SEM; FIG. 7G) and could mold into soft hydrogels with as little as 2 mg/mL or 0.2% w/v (FIG. 7F). Changing a Trp residue to Ala (1-4wa) resulted in an increased solubility and delayed aggregation kinetics (>2 hr), which was consistent with tryptophan's importance in NANOG's self-assembly. However, the 1-4wa peptide displayed more amyloid-like behavior as shown by greater ThT amyloid-bound state (FIG. 7D) and TEM fibril morphologies. The human C-terminal WR repeats (5-8w and 6-8w) and the mouse WR peptides (m1-5w and m6-10w) showed relatively higher solubility and slower aggregation kinetics.

Without being bound by theory, Applicant envisions the presence of charged and ‘structure breaker’ residues in their peptide sequences offset the effects of aggregation-favorable residues. Nevertheless, all WR peptides (including 1w) could form fibrils at higher concentrations (˜0.3-1 mg/mL) and longer incubation times.

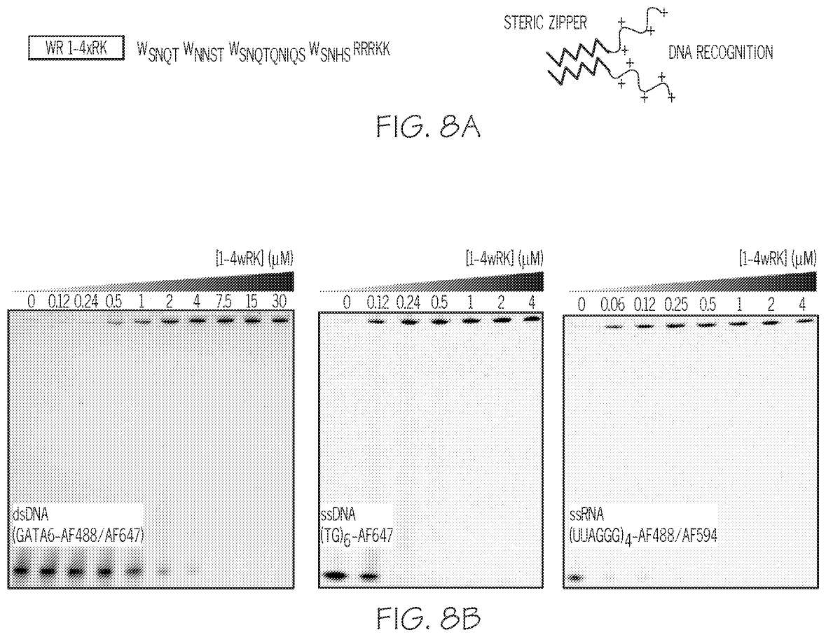

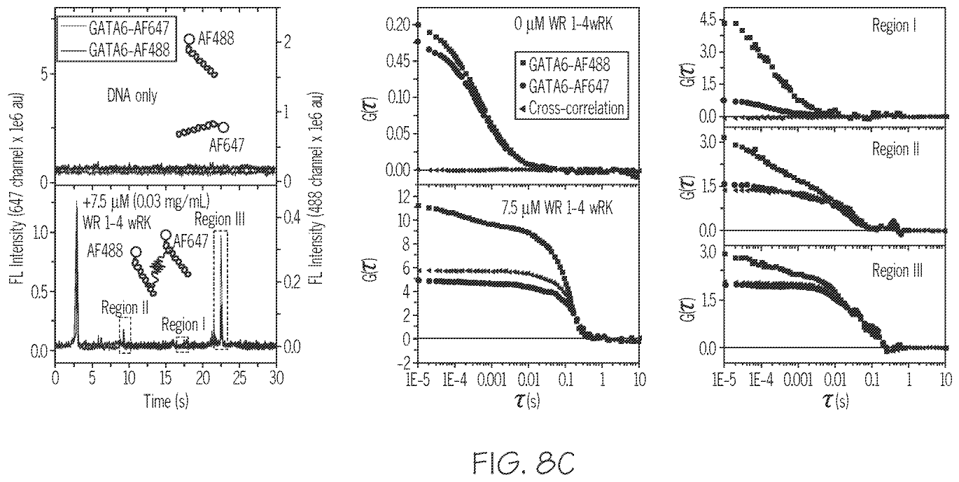

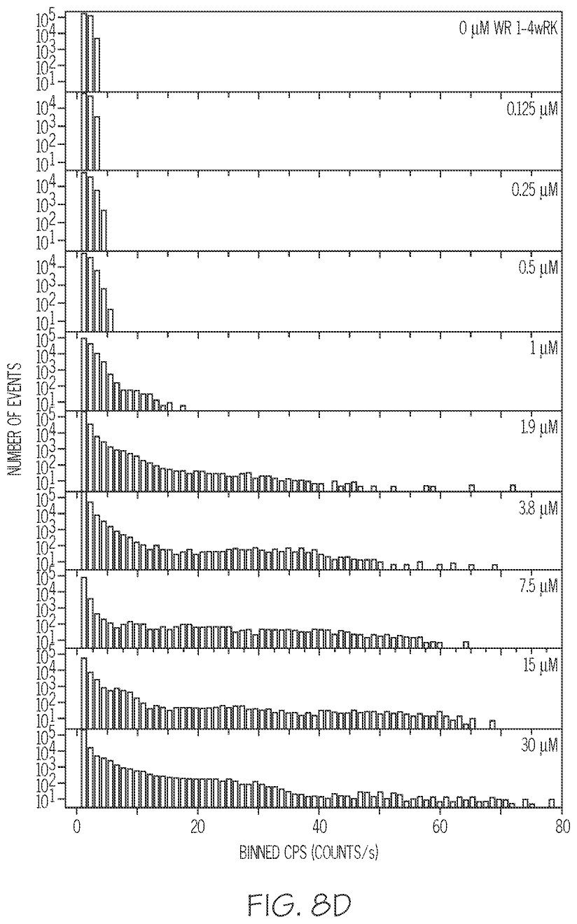

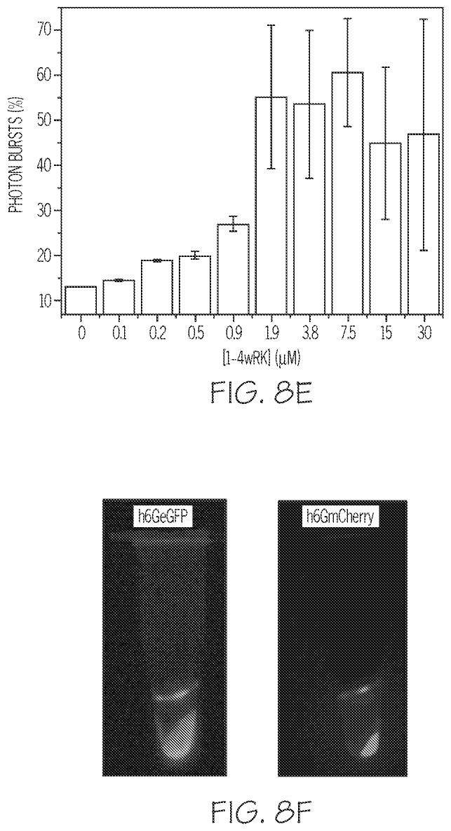

NANOG functions as a master transcription factor and recognizes DNA elements through its basic DNA binding domain (DBD). To test if Applicant could mimic the DNA-binding ability of full-length NANOG with a minimalist peptide, Applicant designed a construct (1-4wRK peptide) with additional basic residues (3 Arg and 2 Lys) that could facilitate nonspecific nucleic acid recognition through known π-cation interactions (FIG. 8A).

Applicant observed nucleic acid interactions by fluorescence electrophoretic mobility shift assays (fEMSA). The 1-4wRK peptide bound double stranded DNA (dsDNA GATA6-AF488/GATA6-AF647) with ˜1 μM affinity (50% free DNA; FIG. 8B), approximating that of full-length NANOG (˜30-60 nM). Moreover, the 1-4wRK peptide was also potent in recognizing single stranded DNA (ssDNA (TG)₆) and RNA (ssRNA (UUAGGG)₄-AF488/AF594). Completely bound peptide: nucleic acid complexes (immobilized in the gel wells) were observed with as little as 60-250 nM or 0.02-0.1 mg/mL peptide (FIG. 8B, middle and right panels).

Applicant also tested the minipeptides' ability to bring together independent DNA molecules in solution. Using Fluorescence Cross-Correlation Spectroscopy (FCCS), Applicant observed an absence of cross-correlation between differentially fluorescently labeled DNAs (GATA6-AF488 and GATA6-AF647) without the peptide. In the presence of the 1-4wRK peptide, Applicant observed photon bursts/spikes (e.g., Region II and III in FIG. 8C) that correlated with high cross-correlation between different fluorescent DNA species (blue curves, FIG. 8C). The slower diffusion observed in the FCCS curves for Region II and III as compared to Region I indicated larger diffusing species co-migrating with the DNAs. Titration with various concentrations of 1-4wRK peptide resulted in cross-correlation with as little as 1 μM peptide, consistent with the fEMSA data.

Applicant used burst analysis to quantify sizes of the oligomeric species. The average fluctuations were assumed to represent monomeric species, and any degree of deviations from average were characterized as the oligomeric states. Applicant observed numerous heterogeneous oligomeric states that increased in molecular sizes with increasing peptide concentration (FIGS. 8D-8E). Because NANOG is also known as a ‘molecular hub’ protein capable of interactions with many proteins with its WR domain, Applicant tested whether the WR peptide mimetics could non-specifically recruit random proteins (e.g., fluorescent proteins h6GeGFP and h6GmCherry for convenient visualization). Both fluorescent proteins were soluble on their own, but in the presence of as little as ˜0.3 mg/mL 1-4wRK, both proteins formed visible precipitates (FIG. 8F).

A protein's residue composition can dictate its physical material properties. For instance, hydrophobic and polar residues contribute to ‘hardening’ and charged residues contribute to ‘softening’. By altering the peptide properties, Applicant tested if they could ‘soften’ the amyloid aggregation mechanism from a direct liquid to solid phase transitions (LSPT) to that with the formation of intermediate mesoscale liquid droplets via liquid-liquid phase separation (LLPS).

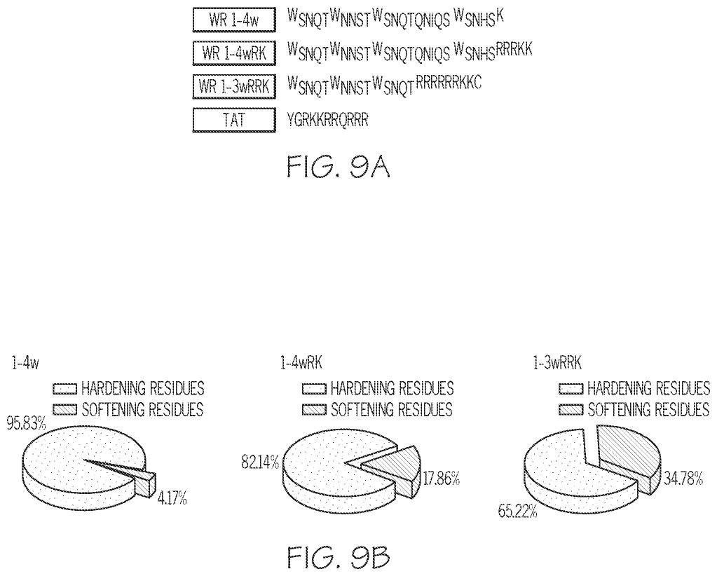

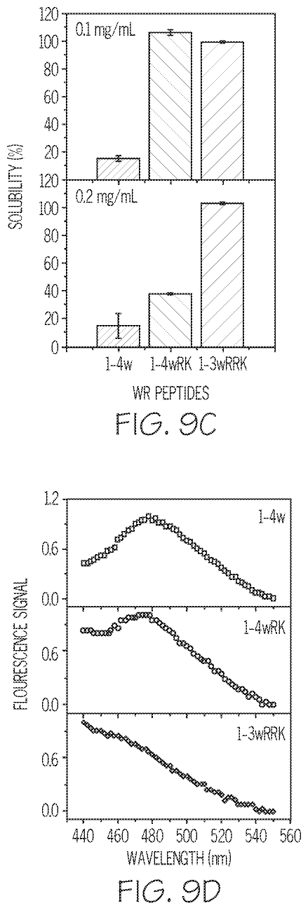

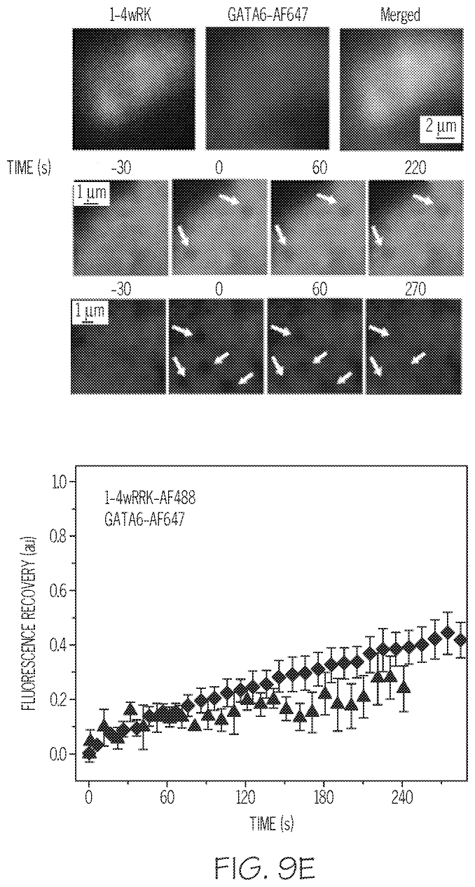

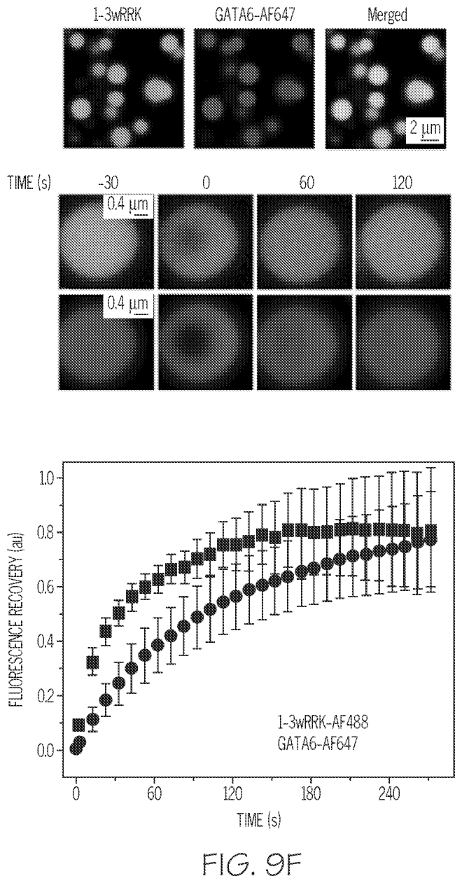

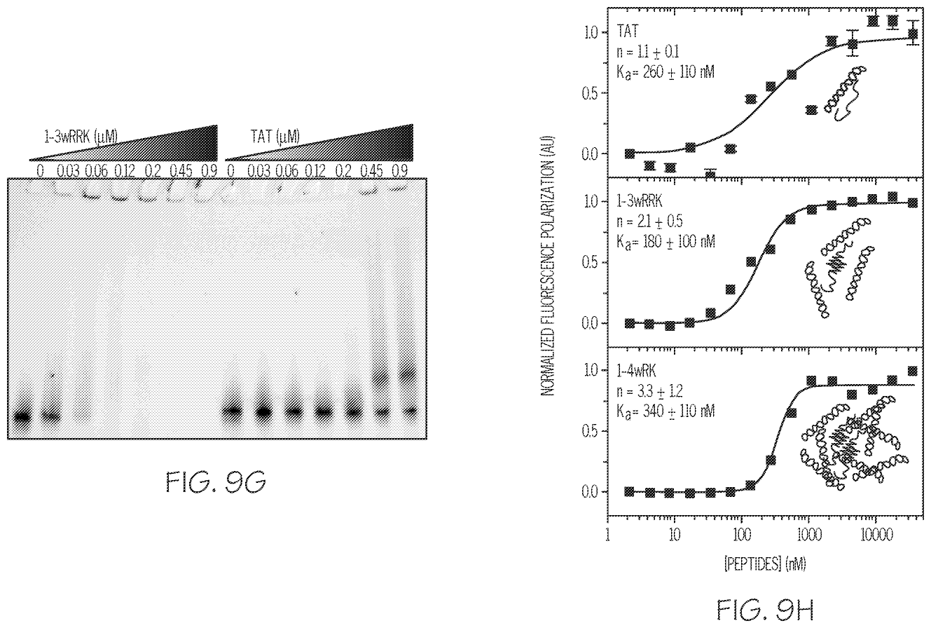

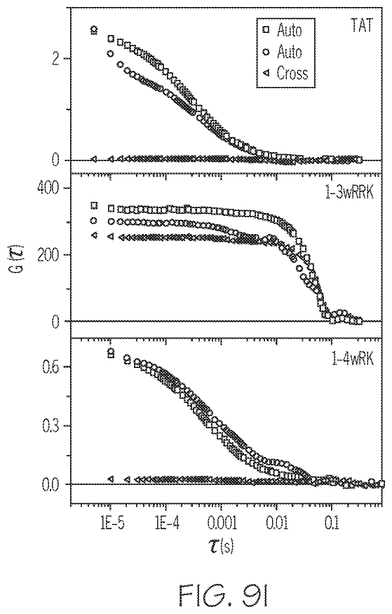

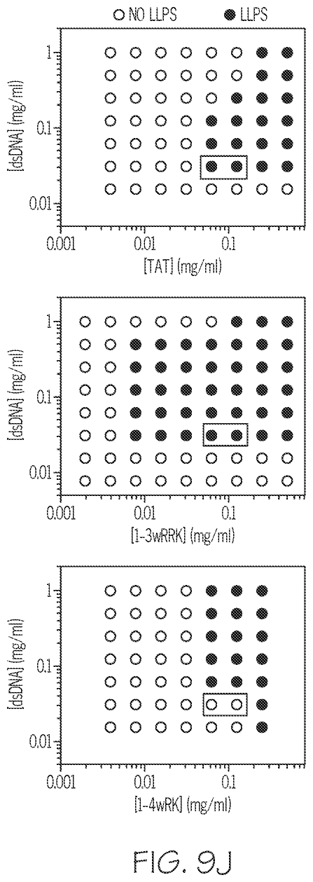

Applicant designed a construct (1-3wRRK) with reduced WR repeats (i.e., ‘hardening’ residues) and increased charged or ‘softening’ residues (FIGS. 9A-9B). Applicant decided on a total of 6 Arg and 3 Lys to directly compare the construct's DNA-binding ability with the well-known cell-penetrating peptide HIV Trans-Activator of Transcription (TAT). By decreasing ‘hardening’ residues and increasing ‘softening’ residues, Applicant was able to increase solubilities and decreased amyloidal propensities of the novel peptides (1-4wRK and 1-3wRRK), as compared to the original highly aggregation-prone 1-4w construct (FIGS. 9C-9D). In particular, the 1-3wRRK peptide was very soluble on its own and only underwent LLPS upon interaction with DNA.

Using fluorescence recovery after photobleaching (FRAP) imaging of 1-4wRK vs. 1-3wRRK peptide complexes with DNA, Applicant confirmed attenuated fluorescence recovery or slower diffusion of 1-4wRK:DNA complexes in irregular clusters compared to 1-3wRRK:DNA in liquid droplets. Thus, Applicant were able to alter the peptide's aggregation behavior from LLPS-independent to LLPS-mediated mechanisms (FIGS. 9E-9F).

Since 1-3wRRK became soluble and much less aggregation-prone on its own, Applicant determined if the WR repeat (steric zipper region) was affected by changes in physical properties and still contributed towards recognizing DNA. Applicant compared its DNA binding capability to TAT and 1-4wRK by EMSA and fluorescence polarization measurements (FIGS. 9G-9H). Applicant observed that 1-3wRRK binds DNA more effectively than 1-4wRK, mostly likely due to the extra basic residues (FIGS. 8B, 9G, and 9H).

1-3wRK bound DNA significantly better than TAT. By EMSA, almost all DNAs were bound at 60 nM or 0.03 mg/mL 1-3wRRK. In contrast, even at 0.9 μM TAT, not all of the DNAs were bound and Applicant observed fewer shifted bands in the fEMSA gel (FIG. 9G). This was further corroborated by fluorescence polarization measurements. Comparing the binding of the three peptides, Applicant found that 1-4wRK had a slightly lower binding constant (Kₐ=340±110 nM) but with higher cooperativity (n=3.3±1.2) among the three peptides (TAT, Kₐ=260±110, n=1.1±0.1; and, 1-3wRRK, Kₐ=180±100 nM, n=2.1±0.5).

Applicant further compared the ability of the peptides to bridge independent DNA molecules by FCCS (FIG. 9I). At 0.9 μM peptides, Applicant observed no cross-correlation for TAT, a small degree of cross-correlation for 1-4wRK, but a large cross-correlation for 1-3wRK.

The significant 1-3wRK DNA-bridging competency was further confirmed by FCCS titration data and burst analysis, where Applicant observed cross-correlation even at ˜30 nM 1-3wRRK. Applicant next investigated whether the peptides' ability to form nano-assemblies also correlated with their capacity to drive mesoscale condensates. Applicant demarcated the LLPS regimes using turbidity assay (UV absorbance at 350 nm; FIG. 8J). The LLPS data (presented in mg/mL concentration unit in FIG. 8J) reflected the behaviors observed at nanoscale level. 1-3wRRK was the most potent in driving DNA: peptide assemblies; LLPS were observed at lower DNA and peptide concentrations. 1-4wRRK was effective at lower concentrations of DNA (red box) but more effective than TAT at higher concentrations of DNA.

All data confirmed that the steric zipper has significant contribution to recruiting DNA at nano-and mesoscales. Essentially, with the 1-3wRRK construct, Applicant was able to take advantage of the steric zipper's enhanced binding cooperativity but with favorable solubility and functional properties (partitioning of DNA and protein GFP). Moreover, the material properties of 1-3wRRK are tunable, and solution phase changes are triggered only with external stimuli (e.g., DNA/protein interaction).

The tunability, simplicity and potency of NANOG minipeptides inspire possibilities for translational applications. Many bio-inspired nanomaterials, such as TAT, mussel or slug adhesives, Sup35 or other prion-based nanomaterials, have various drug/biologics delivery and industrial applications. Applicant performed experiments to test some possibilities.

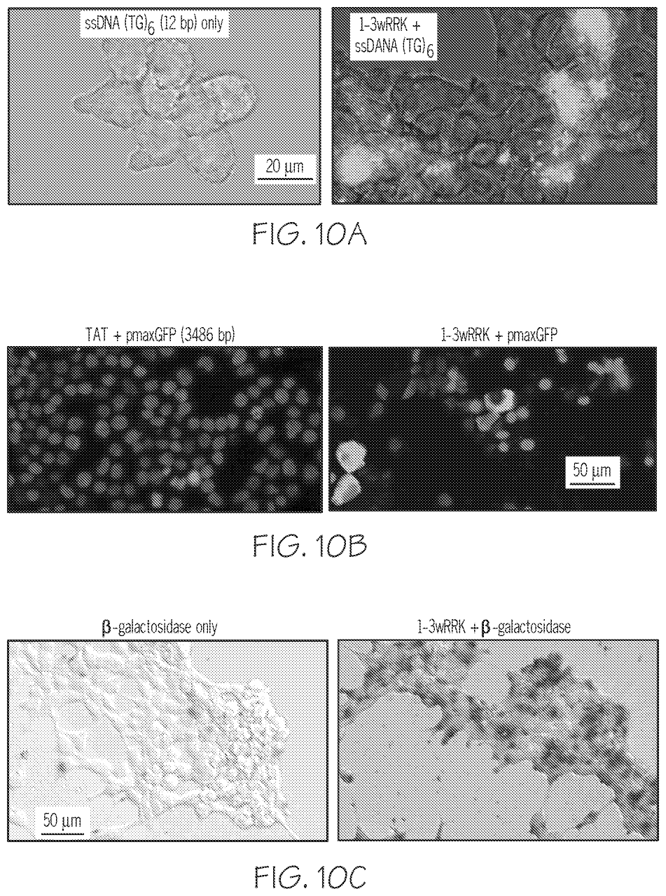

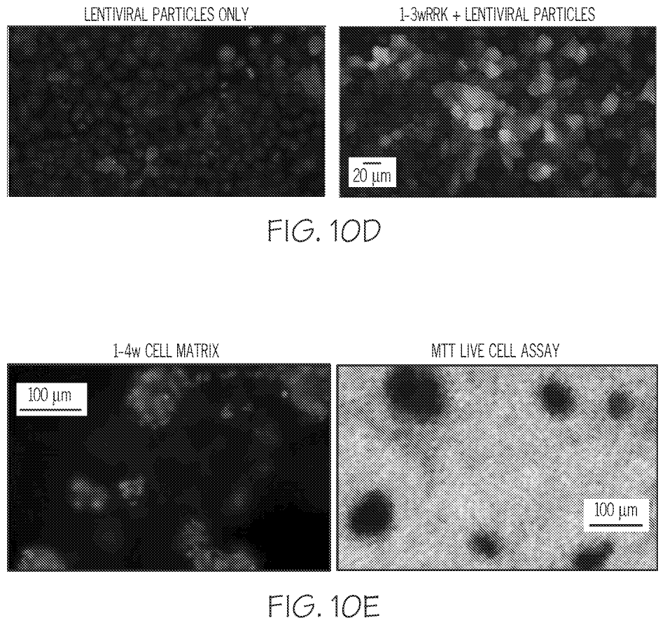

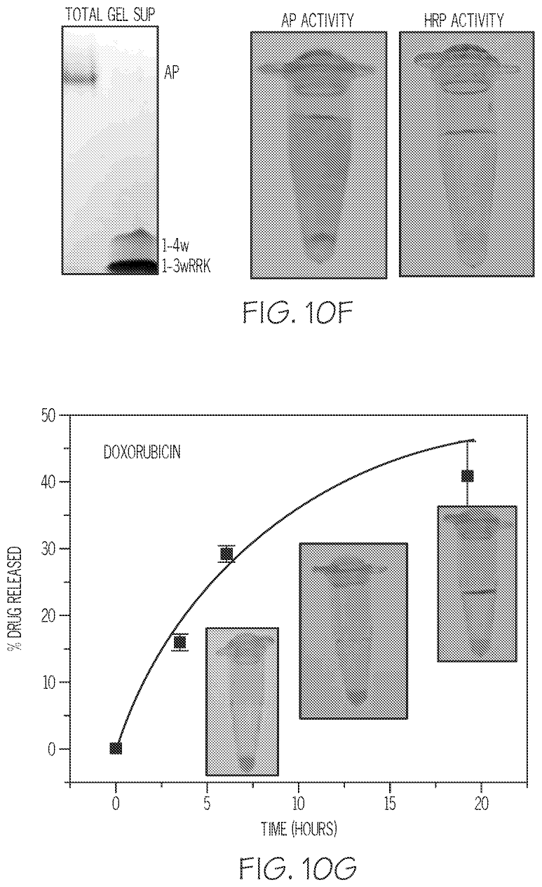

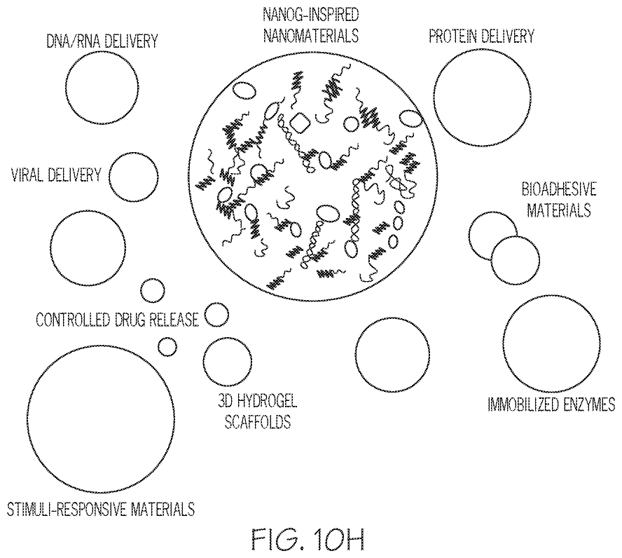

The abilities of 1-4wRK and 1-3wRK to bind and partition nucleic acids and proteins (FIGS. 8F, and 9H-I) at nanoscale and mesoscale levels make them effective delivery vehicles. WR 1-3wRRK readily entered mammalian HEK 293T cells on its own. It could deliver small 12 bp ssDNA (FIG. 10A) or large ˜4 kbp plasmid vectors (FIG. 10B) into cells. Furthermore, it could deliver proteins (e.g., ˜34 kDa β-galactosidase, FIG. 10C) or large lentivirus particles (FIG. 10D).

Another growing demand is the use of hydrogels in pharmaceutical and industrial applications. Peptide-based hydrogels have the advantages of inherent biocompatibility, biodegradability and tunability. For example, Applicant's WR-based hydrogels could provide a three-dimensional scaffold for cellular growth (FIG. 10E), which would be important for the initiation of tissue or organoid cultures.

Other proteins, growth factors, and drugs could be incorporated with hydrogels for a slow controlled release into cells for therapeutic applications. For instance, FIG. 10F showed the slow release (>20 hours) of Doxorubicin, a chemotherapeutic agent embedded in the WR-based hydrogels. Another application is the use of hydrogels for immobilizing enzymes. Applicant's WR-based hydrogels could partition enzymes without the need for cross-linking or complicated conjugation strategies, generating catalytically active solid scaffolds (FIG. 10G).

Moreover, the fibrillation potency of WR-based peptides enables easy handling and manufacturing of nanomaterials without the need for complicated chemical synthesis or conditions for generating fibrils. Furthermore, because Applicant can modulate the material properties through peptide concentrations, diverse combinations of WR peptide-based variants, and/or co-partitioning with drugs and biologics, Applicant can create various types of ‘Nanog-inspired biomaterials’ (FIG. 10H) from nanoscale to macroscale levels with diverse translational applications in therapeutics and biotechnology.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. At least one isolated peptide, wherein the at least one isolated peptide comprises a sequence selected from the group consisting of:
(SEQ ID NO: 1)
WSNQTK;
(SEQ ID NO: 2)
WSNQTWNNSTK;
(SEQ ID NO: 3)
WSNQTWNNSTWSNQTK;
(SEQ ID NO: 4)
WSNQTWNNSTWSNQTQNIQSWSNHSK;
(SEQ ID NO: 5)
ASNQTANNSTASNQTQNIQSWSNHSK;
(SEQ ID NO: 6)
WCTQSWNNQAWNSPFYNCGEESK;
(SEQ ID NO: 7)
WNTQTWCTQSWNNQAWNSPFYNCGEESK;
(SEQ ID NO: 8)
WGSQTWTNPTWSSQTWTNPTWNNQTK;
(SEQ ID NO: 9)
WTNPTWSSQAWTAQSWNGQPWNAAPK;
(SEQ ID NO: 10)
WSNQTWNNSTWSNQTQNIQSWSNHSRRRKK;
(SEQ ID NO: 11)
WSNQTWNNSTWSNQTRRRRRRKKC;
(SEQ ID NO: 12)
WSNQTWNNSTWSNQTQNIQSWSNHSWNTQTWCTQSWNNQAWNSPFYN;
(SEQ ID NO: 13)
WSNQTWNNSTWSNQTQNIQSWSNHSWNTQTWCTQSWNNQAWNSPFYNCGE
ESM;
(SEQ ID NO: 14)
WGSQTWTNPTWSSQTWTNPTWNNQTKWTNPTWSSQAWTAQSWNGQPWNAA
PK;
derivatives thereof; analogs thereof; homologs thereof; and combinations thereof.

2. The at least isolated peptide of claim 1, wherein the at least one isolated peptide comprises a plurality of isolated peptides, wherein each of the plurality of isolated peptides comprises a sequence selected from the group consisting of SEQ ID NOS: 1-14, derivatives thereof, analogs thereof, homologs thereof, and combinations thereof.

3. The at least one isolated peptide of claim 1, wherein the at least one isolated peptide is in aggregated form.

4. The at least one isolated peptide of claim 1, wherein the at least one isolated peptide is in fibrillated form.

5. The at least one isolated peptide of claim 1, wherein the at least one isolated peptide is in the form of a three-dimensional hydrogel.

6. The at least one isolated peptide of claim 1, wherein the at least one isolated peptide comprises an analog or homolog of any one of SEQ ID NOS: 1-14, wherein the analog or homolog is at least 65% identical to any of SEQ ID NOS: 1-14.

7. (canceled)

8. The at least one isolated peptide of claim 1, wherein the at least one isolated peptide comprises a derivative of any one of SEQ ID NOS: 1-14, wherein the derivative comprises one or more amino acid moieties derivatized with one or more functional groups, wherein the one or more functional groups are positioned on amino acid backbones, R groups, or combinations thereof, and wherein the one or more functional groups are selected from the group consisting of alkanes, alkenes, ethers, alkynes, alkoxyls, aldehydes, carboxyls, hydroxyls, hydrogens, sulfurs, phenyls, cyclic rings, aromatic rings, heterocyclic rings, linkers, or combinations thereof.

9. (canceled)

10. The at least one isolated peptide of claim 1, wherein the at least one isolated peptide comprises SEQ ID NO: 1 or a sequence with at least 65% sequence identity to SEQ ID NO: 1.

11. The at least one isolated peptide of claim 1, wherein the at least one isolated peptide comprises SEQ ID NO: 2 or a sequence with at least 65% sequence identity to SEQ ID NO: 2.

12. The at least one isolated peptide of claim 1, wherein the at least one isolated peptide comprises SEQ ID NO: 3 or a sequence with at least 65% sequence identity to SEQ ID NO: 3.

13. The at least one isolated peptide of claim 1, wherein the at least one isolated peptide comprises SEQ ID NO: 4 or a sequence with at least 65% sequence identity to SEQ ID NO: 4.

14. The at least one isolated peptide of claim 1, wherein the at least one isolated peptide comprises SEQ ID NO: 5 or a sequence with at least 65% sequence identity to SEQ ID NO: 5.

15. The at least one isolated peptide of claim 1, wherein the at least one isolated peptide comprises SEQ ID NO: 6 or a sequence with at least 65% sequence identity to SEQ ID NO: 6.

16. The at least one isolated peptide of claim 1, wherein the at least one isolated peptide comprises SEQ ID NO: 7 or a sequence with at least 65% sequence identity to SEQ ID NO: 7.

17. The at least one isolated peptide of claim 1, wherein the at least one isolated peptide comprises SEQ ID NO: 8 or a sequence with at least 65% sequence identity to SEQ ID NO: 8.

18. The at least one isolated peptide of claim 1, wherein the at least one isolated peptide comprises SEQ ID NO: 9 or a sequence with at least 65% sequence identity to SEQ ID NO: 9.

19. The at least one isolated peptide of claim 1, wherein the at least one isolated peptide comprises SEQ ID NO: 10 or a sequence with at least 65% sequence identity to SEQ ID NO: 10.

20. The at least one isolated peptide of claim 1, wherein the at least one isolated peptide comprises SEQ ID NO: 11 or a sequence with at least 65% sequence identity to SEQ ID NO: 11.

21. The at least one isolated peptide of claim 1, wherein the at least one isolated peptide comprises SEQ ID NO: 12 or a sequence with at least 65% sequence identity to SEQ ID NO: 12.

22. The at least one isolated peptide of claim 1, wherein the at least one isolated peptide comprises SEQ ID NO: 13 or a sequence with at least 65% sequence identity to SEQ ID NO: 13.

23. The at least one isolated peptide of claim 1, wherein the at least one isolated peptide comprises SEQ ID NO: 14 or a sequence with at least 65% sequence identity to SEQ ID NO: 14.

24. (canceled)

25. The at least one isolated peptide of claim 1, wherein the at least one isolated peptide is associated with one or more materials selected from the group consisting of small molecules, drugs, proteins, enzymes, catalysts, virus particles, nucleotides, DNA, RNA, plasmids, and combinations thereof.

26-27. (canceled)

28. A method of delivering at least one isolated peptide into cells, said method comprising:
exposing the cells to the at least one isolated peptide, a nucleotide sequence that expresses the at least one isolated peptide, or combinations thereof wherein the at least one isolated peptide comprises a sequence selected from the group consisting of:
(SEQ ID NO: 1)
WSNQTK;
(SEQ ID NO: 2)
WSNQTWNNSTK;
(SEQ ID NO: 3)
WSNQTWNNSTWSNQTK;
(SEQ ID NO: 4)
WSNQTWNNSTWSNQTQNIQSWSNHSK;
(SEQ ID NO: 5)
ASNQTANNSTASNQTQNIQSWSNHSK;
(SEQ ID NO: 6)
WCTQSWNNQAWNSPFYNCGEESK;
(SEQ ID NO: 7)
WNTQTWCTQSWNNQAWNSPFYNCGEESK;
(SEQ ID NO: 8)
WGSQTWTNPTWSSQTWTNPTWNNQTK;
(SEQ ID NO: 9)
WTNPTWSSQAWTAQSWNGQPWNAAPK;
(SEQ ID NO: 10)
WSNQTWNNSTWSNQTQNIQSWSNHSRRRKK;
(SEQ ID NO: 11)
WSNQTWNNSTWSNQTRRRRRRKKC;
(SEQ ID NO: 12)
WSNQTWNNSTWSNQTQNIQSWSNHSWNTQTWCTQSWNNQAWNSPFYN;
(SEQ ID NO: 13)
WSNQTWNNSTWSNQTQNIQSWSNHSWNTQTWCTQSWNNQAWNSPFYNCGE
ESM;
(SEQ ID NO: 14)
WGSQTWTNPTWSSQTWTNPTWNNQTKWTNPTWSSQAWTAQSWNGQPWNAA
PK;
derivatives thereof; analogs thereof; homologs thereof; and combinations thereof.

29. The method of claim 28, wherein the exposing comprises exposing the cells to the at least one isolated peptide of claim 28, wherein the exposing results in the delivery of the at least one isolated peptide into the cells.

30. The method of claim 28, wherein the exposing comprises exposing the cells to a nucleotide sequence that expresses the at least one isolated peptide of claim 28, wherein the exposing results in the expression of the at least one isolated peptide in the cells.

31. The method of any one of claims 28, wherein the cells comprise human cells.

32. The method of claim 28, wherein the exposing occurs in vitro.

33. (canceled)

34. The method of claim 28, wherein the exposing occurs in vivo in a subject.

35. The method of claim 34, wherein the exposing comprises administering to the subject the at least one isolated peptide, the nucleotide sequence that expresses the at least one isolated peptide, or combinations thereof.

36. (canceled)

37. The method of claim 34, wherein the at least one isolated peptide is used to treat or prevent a disorder or disease in the subject.

38. The method of claim 28, wherein the cells are exposed to the at least one isolated peptide of claim 28, wherein the at least one isolated peptide is associated with one or more materials, and wherein the method is utilized to deliver the one or more materials into the cells, wherein the one or more materials is selected from the group consisting of small molecules, drugs, proteins, enzymes, catalysts, virus particles, nucleotides, DNA, RNA, plasmids, and combinations thereof.

39. (canceled)


SEQUENCE LISTING (28 sequences)

SEQ ID NO: 1 - protein, 6 aa, synthetic construct
WSNQTK

SEQ ID NO: 2 - protein, 11 aa, synthetic construct
WSNQTWNNSTK

SEQ ID NO: 3 - protein, 16 aa, synthetic construct
WSNQTWNNSTWSNQTK

SEQ ID NO: 4 - protein, 26 aa, synthetic construct
WSNQTWNNSTWSNQTQNIQSWSNHSK

SEQ ID NO: 5 - protein, 26 aa, synthetic construct
ASNQTANNSTASNQTQNIQSWSNHSK

SEQ ID NO: 6 - protein, 23 aa, synthetic construct
WCTQSWNNQAWNSPFYNCGEESK

SEQ ID NO: 7 - protein, 28 aa, synthetic construct
WNTQTWCTQSWNNQAWNSPFYNCGEESK

SEQ ID NO: 8 - protein, 26 aa, synthetic construct
WGSQTWTNPTWSSQTWTNPTWNNQTK

SEQ ID NO: 9 - protein, 26 aa, synthetic construct
WTNPTWSSQAWTAQSWNGQPWNAAPK

SEQ ID NO: 10 - protein, 30 aa, synthetic construct
WSNQTWNNSTWSNQTQNIQSWSNHSRRRKK

SEQ ID NO: 11 - protein, 24 aa, synthetic construct
WSNQTWNNSTWSNQTRRRRRRKKC

SEQ ID NO: 12 - protein, 47 aa, synthetic construct
WSNQTWNNSTWSNQTQNIQSWSNHSWNTQTWCTQSWNNQAWNSPFYN

SEQ ID NO: 13 - protein, 53 aa, synthetic construct
WSNQTWNNSTWSNQTQNIQSWSNHSWNTQTWCTQSWNNQAWNSPFYNCGEESM

SEQ ID NO: 14 - protein, 52 aa, synthetic construct
WGSQTWTNPTWSSQTWTNPTWNNQTKWTNPTWSSQAWTAQSWNGQPWNAAPK

SEQ ID NO: 15 - protein, 47 aa, synthetic construct; identical to SEQ ID NO: 12

SEQ ID NO: 16 - protein, 7 aa, Homo sapiens
GNNQQNY

SEQ ID NO: 17 - protein, 7 aa, Homo sapiens
TQNIQSW

SEQ ID NO: 18 - protein, 6 aa, Homo sapiens
SNQNNF

SEQ ID NO: 19 - protein, 5 aa, Homo sapiens
SNQTW

SEQ ID NO: 20 - protein, 52 aa, Homo sapiens
WSNQTWNNSTWSNQTQNIQSWSNHSWNTQTWCTQSWNNQAWNSPFYNCGEES

SEQ ID NO: 21 - protein, 50 aa, Mus famulus
WGSQTWTNPTWSSQTWTNPTWNNQTWTNPTWSSQAWTAQSWNGQPWNAAP

SEQ ID NO: 22 - protein, 30 aa, synthetic construct; identical to SEQ ID NO: 10

SEQ ID NO: 23 - DNA, 12 bp, synthetic construct
  source 1..12: mol_type = other DNA
tgtgtgtgtgtg

SEQ ID NO: 24 - RNA, 24 nt, synthetic construct
  source 1..24: mol_type = other RNA
ttagggttagggttagggttaggg

SEQ ID NO: 25 - protein, 26 aa, synthetic construct
WSNQTWNNSTWSNQTQNIQSWSNHSK

SEQ ID NO: 26 - protein, 30 aa, synthetic construct; identical to SEQ ID NO: 10

SEQ ID NO: 27 - protein, 24 aa, synthetic construct
WSNQTWNNSTWSNQTRRRRRRKKC

SEQ ID NO: 28 - protein, 11 aa, synthetic construct
YGRKKRRQRRR